(19) United States
(12) Patent Application Publication
(10) Pub. No.: US 2017/0042989 A1
(43) Pub. Date: Feb. 16, 2017

(54) PHOTOSENSITIZED TRYPANOSOMATIDS AND VACCINE COMPOSITIONS THEREOF

(71) Applicants: ROSALIND FRANKLIN UNIVERSITY OF MEDICINE AND SCIENCE, North Chicago, IL (US); THE CHINESE UNIVERSITY OF HONG KONG, Hong Kong SAR (CN)

(72) Inventors: Kwang Poo Chang, North Chicago, IL (US); Bala Kolli, North Chicago, IL (US); Dennis K.P. Ng, Hong Kong (CN)

(21) Appl. No.: 15/234,378

(22) Filed: Aug. 11, 2016

Related U.S. Application Data
(60) Provisional application No. 62/204,188, filed on Aug. 12, 2015.

(51) Int. Cl. A61K 39/005; A61K 41/00

(52) U.S. Cl. CPC A61K 39/005; A61K 41/0071; A61K 2039/521

(57) ABSTRACT

Disclosed herein are methods and compositions for the delivery of polypeptides, including vaccine candidate polypeptides, into mammalian cells comprising the use of photosensitized trypanosomatid organisms. Also disclosed are methods of treatment of trypanosomatid infections comprising administering phthalocyanine compounds or phthalocyanine-treated trypanosomatid microorganisms as vaccines, as well as and polypeptide delivery vectors comprising phthalocyanine-treated trypanosomatid microorganisms.

### CROSS-REFERENCE TO RELATED APPLICATIONS

This application claims the benefit of U.S. Provisional Application No. 62/204,188, filed Aug. 12, 2015, the disclosure of which is hereby incorporated by reference in its entirety.

### BACKGROUND

The disclosure relates to delivery of therapeutic polypeptides into cells. Proteins and peptides have the potential to be valuable prophylactic and therapeutic agents in humans and other animal subjects. However, because proteins and peptides are larger and more complex than conventional organic and inorganic drug molecules, the formulation and delivery of such agents present unique problems. In this regard, potentially beneficial protein and peptide drugs typically require the maintenance of their integrity in order to be efficacious with regard to their desired biological properties against the intended targets. A protein's integrity can be altered by any of the numerous protein degradation pathways present in the body.

While there have been extensive and ongoing research efforts focused on novel ways to successfully deliver protein and peptide drugs to their intended targets, there remains a need for effective delivery techniques for polypeptides.

The disclosure also relates to treatments for trypanosomatid infections, such as leishmaniasis. Leishmaniasis, also spelled leishmaniosis, is a disease caused by protozoan trypanosomatid parasites of the genus Leishmania and is spread by the bite of certain types of sandflies. Typically, disease presents in one of three ways: cutaneous, mucocutaneous, or visceral leishmaniasis. The cutaneous form presents with skin ulcers, while the mucocutaneous form presents with ulcers of the skin, mouth, and nose, and the visceral form starts with skin ulcers and then later presents with fever, low red blood cells, and enlarged spleen and liver. Infections in humans are caused by more than 20 species of Leishmania.

Leishmaniasis occurs in 88 tropical and subtropical countries, including through much of the Americas from northern Argentina to Texas, much of Asia, Africa, Mediterranean basin and the Middle East. About 350 million people live in these affected areas. As many as 12 million people are affected worldwide, with 1.5-2.0 million new cases each year.

The treatment is determined by where the disease was acquired, the species of Leishmania, and the type of infection. For visceral leishmaniasis in India, South America, and the Mediterranean, liposomal amphotericin B is the recommended treatment and is often used as a single dose. Rates of cure with a single dose of amphotericin have been reported as 95%. In India, almost all infections are resistant to pentavalent antimony compounds (antimonials), whereas in Africa, a combination of pentavalent antimonials and paromomycin is recommended. These, however, can have significant side effects. Miltefosine, an oral medication, is effective against both visceral and cutaneous leishmaniasis. Side effects are generally mild, though it can cause birth defects if taken within 3 months of getting pregnant, and it is not effective against L. major or L. braziliensis.

There is thus a need for improved control (prevention and treatment) for leishmaniasis, as well as for other diseases caused by trypanosomatid infections.

### SUMMARY

This disclosure provides certain advantages and advancements over the prior art, in particular, vaccines and polypeptide delivery vectors comprising phthalocyanine-treated trypanosomatid microorganisms, as well as methods for treating trypanosomatid infections comprising administering phthalocyanine compounds.

In one aspect, the disclosure provides compositions for delivering polypeptides into mammalian cells, the compositions comprising a trypanosomatid that has been treated with a compound of formula (I):

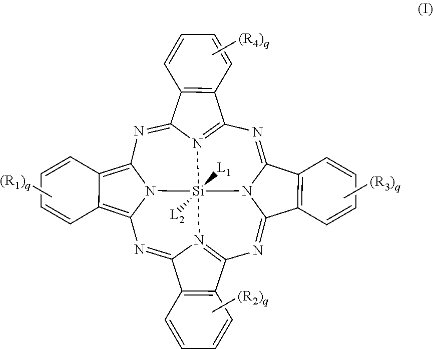

or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
   each R is independently —[C₁-C₆ alkylene-O]ₘ—R′ or —[C₁-C₆ alkylene-NR″]ₙ—R′,
   each m and n are independently an integer selected from 1 to 20,
   each R′ is independently selected from H and C₁-C₆ alkyl,
   each R″ is independently selected from H and C₁-C₆ alkyl;
   each q is independently an integer selected from 0, 1, and 2; and
   R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂;

wherein the trypanosomatid expresses the polypeptide.

In some embodiments, the trypanosomatid has been transgenically modified to express a cDNA sequence encoding the polypeptide. In some embodiments, the polypeptide is a vaccine candidate. In some embodiments, the trypanosomatid is capable of entering the mammalian cell. In some embodiments, the trypanosomatid has been exposed to light to inactivate the trypanosomatid following treatment of the trypanosomatid with the compound of formula (I). In some embodiments, the trypanosomatid is a Leishmania sp.

In some embodiments, the mammalian cell is a mammalian immune cell. In some embodiments, the mammalian immune cell is an antigen-presenting cell. In some embodiments, the mammalian immune cell is a dendritic cell or a macrophage or a B cell.

In another aspect, the disclosure provides methods for delivering a polypeptide to a cell of a mammalian subject, the methods comprising: (a) providing a trypanosomatid that expresses the polypeptide; (b) contacting the cell with the trypanosomatid by administering the trypanosomatid to the mammalian subject; (c) administering to the subject a photosensitizer, wherein the photosensitizer is a compound of formula (I):

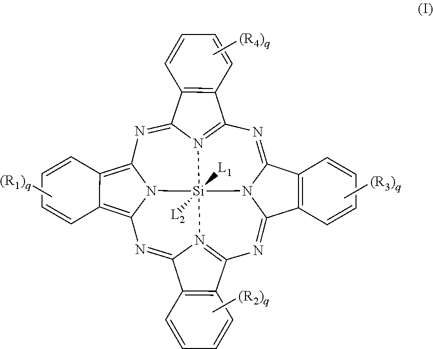

or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
   each R is independently —[C₁-C₆ alkylene-O]ₘ—R′ or —[C₁-C₆ alkylene-NR″]ₙ—R′,
   each m and n are independently an integer selected from 1 to 20,
   each R′ is independently selected from H and C₁-C₆ alkyl,
   each R″ is independently selected from H and C₁-C₆ alkyl;
   each q is independently an integer selected from 0, 1, and 2; and
   R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂; and

(d) exposing the subject to light to inactivate the trypanosomatid.

In another aspect, the disclosure provides methods for delivering a polypeptide to a cell of a mammalian subject, the methods comprising: (a) providing a trypanosomatid that expresses the polypeptide; (b) treating the trypanosomatid with a photosensitizer to produce a carrier, wherein the photosensitizer is a compound of formula (I):

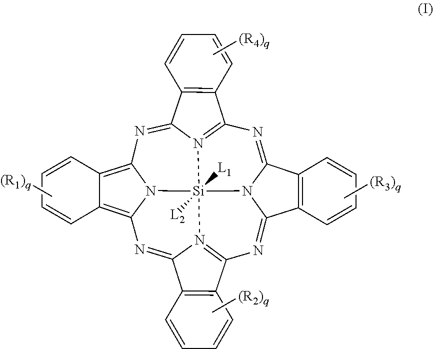

or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
   each R is independently —[C₁-C₆ alkylene-O]ₘ—R′ or —[C₁-C₆ alkylene-NR″]ₙ—R′,
   each m and n are independently an integer selected from 1 to 20,
   each R′ is independently selected from H and C₁-C₆ alkyl,
   each R″ is independently selected from H and C₁-C₆ alkyl;
   each q is independently an integer selected from 0, 1, and 2; and
   R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂;
      (c) contacting the cell with the carrier by administering the carrier to the mammalian subject; and
      (d) exposing the subject to light to inactivate the carrier.

In another aspect, the disclosure provides methods for delivering a polypeptide to a cell of a mammalian subject, the methods comprising: (a) providing a trypanosomatid that expresses the polypeptide; (b) treating the trypanosomatid with a photosensitizer to produce a carrier, wherein the photosensitizer is a compound of formula (I):

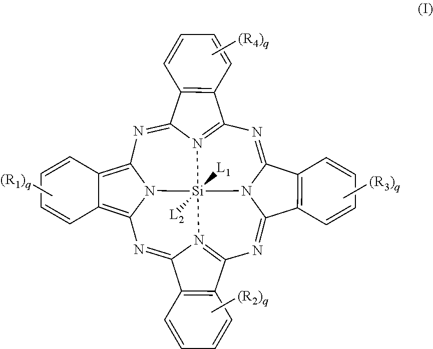

or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
   each R is independently —[C₁-C₆ alkylene-O]ₘ—R′ or —[C₁-C₆ alkylene-NR″]ₙ—R′,
   each m and n are independently an integer selected from 1 to 20,
   each R′ is independently selected from H and C₁-C₆ alkyl,
   each R″ is independently selected from H and C₁-C₆ alkyl;
   each q is independently an integer selected from 0, 1, and 2; and
   R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂;
      (c) exposing the carrier to light to inactivate the carrier; and (d) contacting the cell with the inactivated carrier by administering the carrier to the mammalian subject.

In some embodiments of the above methods, the trypanosomatid has been transgenically modified to express a cDNA sequence encoding the polypeptide. In some embodiments, the polypeptide is a vaccine candidate.

In some embodiments, the wavelength of the light is between about 600 and 700 nm. In some embodiments, the wavelength of the light is more than about 650 nm. In some embodiments, the trypanosomatid is a Leishmania sp. trypanosomatid. In some embodiments, the cell is an immune cell. In some embodiments, the immune cell is an antigen-presenting cell. In some embodiments, the immune cell is a dendritic cell or a macrophage.

In another aspect, the disclosure provides methods of treating a trypanosomatid infection in a subject, the method comprising administering to the subject a therapeutically effective amount of a compound of formula (I):

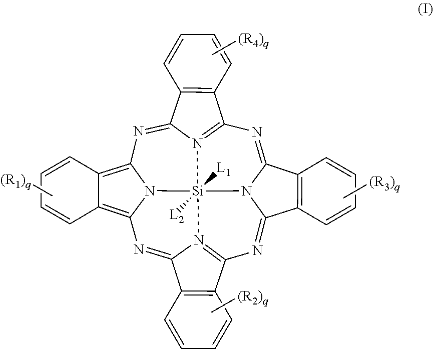

or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
   each R is independently —[C₁-C₆ alkylene-O]ₘ—R′ or —[C₁-C₆ alkylene-NR″]ₙ—R′,
   each m and n are independently an integer selected from 1 to 20,
   each R′ is independently selected from H and C₁-C₆ alkyl,
   each R″ is independently selected from H and C₁-C₆ alkyl;
   each q is independently an integer selected from 0, 1, and 2; and
   R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂.

In some embodiments, the methods further comprise exposing the subject to light following administration of the compound of formula (I). In some embodiments, the wavelength of the light is between about 600 and 700 nm. In some embodiments, the wavelength of the light is more than about 650 nm.

In some embodiments, the compound of formula (I) is administered to the subject topically, intramuscularly, subcutaneously, intravenously, parenterally, enterally, dermally, transbuccally, or intranasally. In some embodiments, the infection is a cutaneous, mucocutaneous, or visceral infection. In some embodiments, the compound of formula (I) is administered to the subject in an in vitro vaccination procedure by: (a) treating antigen-presenting cells from the subject with the compound of formula (I) in vitro; and (b) administering the treated antigen-presenting cells to the subject.

In another aspect, the disclosure provides methods of deactivating a trypanosomatid, the methods comprising: (a) treating a live trypanosomatid with a compound of formula (I):

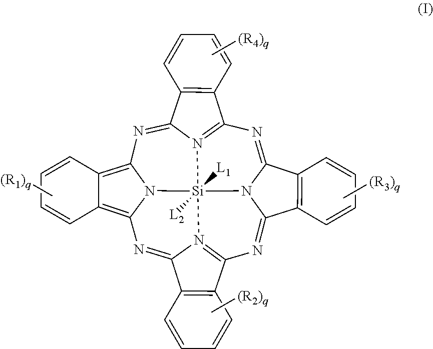

or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
   each R is independently —[C₁-C₆ alkylene-O]ₘ—R′ or —[C₁-C₆ alkylene-NR″]ₙ—R′,
   each m and n are independently an integer selected from 1 to 20,
   each R′ is independently selected from H and C₁-C₆ alkyl,
   each R″ is independently selected from H and C₁-C₆ alkyl;
   each q is independently an integer selected from 0, 1, and 2; and
   R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂; and
      (b) exposing the treated trypanosomatid to light in the presence of normally present atmospheric oxygen to deactivate the trypanosomatid.

In some embodiments, the light is white light. In some embodiments, the light is red light. In some embodiments, the wavelength of the light is between about 600 and 700 nm. In some embodiments, the wavelength of the light is more than about 650 nm. In some embodiments, the trypanosomatid is Leishmania.

In another aspect, the disclosure provides vaccine compositions comprising a deactivated trypanosomatid prepared by the above methods and a suitable pharmaceutical carrier.

In another aspect, the disclosure provides methods for inducing an immune response against a trypanosomatid in a mammalian subject comprising administering to the subject a vaccine composition as disclosed herein. In another aspect, the disclosure provides methods for inducing an immune response against Leishmania in a subject comprising administering to the subject a vaccine composition as disclosed herein, wherein the trypanosomatid is Leishmania.

In some embodiments of any of the methods and/or compositions disclosed herein, each q in formula (I) is an integer selected from 0 and 1. In some embodiments, each q in formula (I) is 0, as in the following formula:

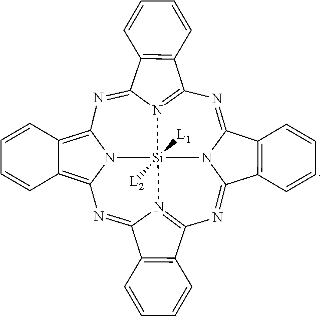

In some embodiments, L₁ and L₂ are independently selected from —O(C₁-C₆ alkyl), —O(C₁-C₆ alkenyl), —O(C₁-C₆ alkynyl), and —OR. In some embodiments, L₁ and L₂ are independently selected from —O(C₁-C₆ alkyl) and —OR. In some embodiments, L₁ and L₂ are independently —OR. In some embodiments, each R is independently —[C₁-C₆ alkylene-NR″]ₙ—R′, or -[ethylene-NR″]ₙ—R′, or -[propylene-NR″]ₙ—R′. In some embodiments, n is an integer selected from 1 to 6, or from 1 to 5, or from 1 to 4, or from 1 to 3, or n is 1, 2, or 3, or n is 1, or n is 2, or n is 3. In some embodiments, R″ is hydrogen or methyl, or R″ is hydrogen. In some embodiments, R′ is hydrogen or methyl, or R′ is hydrogen, or R′ is methyl. In some embodiments, L₁ and L₂ are independently selected from:

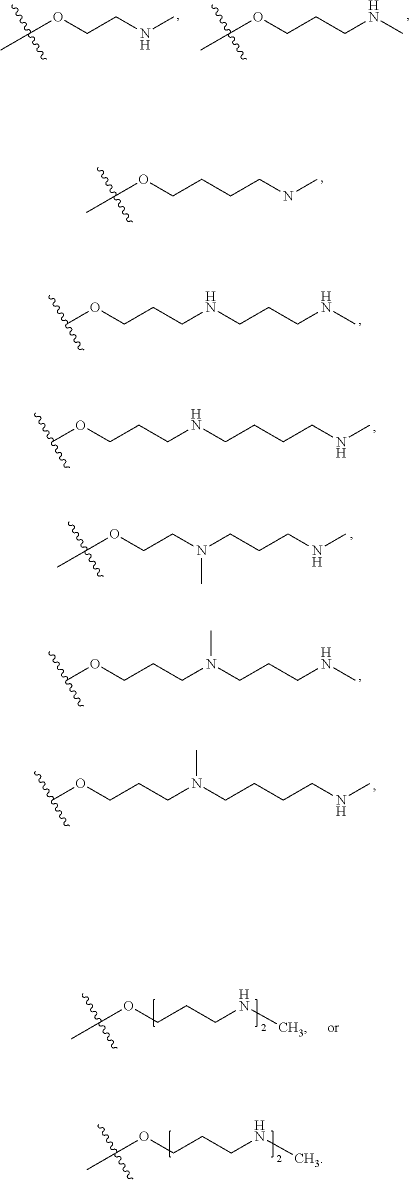

In some embodiments, L₁ and L₂ are independently selected from:

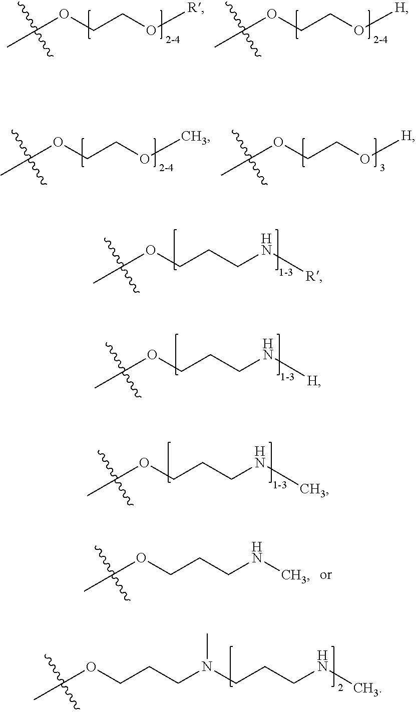

In some embodiments, L₁ and L₂ are independently selected from:

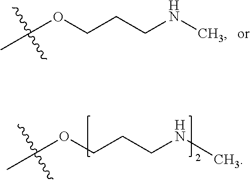

In some embodiments, L₁ and L₂ are the same. In some embodiments, the compound of formula (I) is:

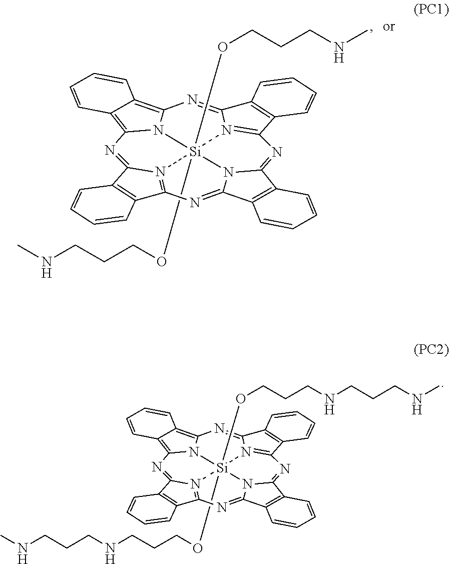

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, in which:

FIG. 1 shows chemical structures of four phthalocyanine conjugates-PC1, PC2, PC3, and PC4.

FIGS. 2A-2D show L. tropica EP41 stage-specific virulence factors and differential infectivity. (A) Western blot analysis of gp63 (leishmanolysin). (B) Cysteine protease activities in native SDS-PAGE gel with gelatin as the substrate; down-regulation of gp63 and up-regulation of cysteine proteases was observed in Pm-to-AxAm (promastigote-to-amastigote) differentiation, as expected. (C) Differential infectivity of Pm vs AxAm to J774 macrophages in vitro, as determined by quantitation of their intracellular replication; left: total # of Leishmania per culture; right: total # of J774 macrophages/culture. (D) Footpad lesions on BALB/c mice after Leishmania tropica infection; footpad swelling was more pronounced with AxAm (upper panel) than with Pm (lower panel).

FIG. 3 shows fluorescent micrographs of Leishmania tropica, showing uptake of amino-phthalocyanine conjugate (PC2) by both stages in vitro. Left panel: Promastigotes A-C/A′-C′; Right panel: Axenic amastigotes D-F/D′-F′. A/A′, D/D′: phase contrast; B/B′, E/E′: Cy5 fluorescence for phthalocyanine PC2; C/C′, F/F′: merged images. Cells were loaded in the dark with 1 μM PC2 for ˜16 hours and washed once before imaging. Short arrow: PC-positive endosomes near flagellar pockets; long arrow: PC-positive filamentous multi-vesicular bodies and endosomes distant from the flagellar pocket.

FIG. 4 shows results of a fluorimetric assay for amino-phthalocyanines extracted from L. tropica after exposure to PC1/PC2, showing their uptake following saturation kinetics. Horizontal axis: time periods in days for sample collections after incubation of promastigotes with PC1 and PC2 in the dark for ˜24 hours. Vertical axis: amounts of PCs extracted from 10⁷ PC-loaded cells taken at different times. The fluorescence readings of cell-associated PCs follow saturation kinetics with times of incubation within each experimental set.

FIG. 5 shows phase contrast and fluorescence micrographs of L. tropica, showing PC1- and PC2-mediated photo-inactivation of its promastigote stage. Shown are the images of: [A] GFP-transfected promastigotes under phase contrast; [B] FITC filter for GFP fluorescence; [C] Cy5 filter for phthalocyanine fluorescence; and [D] Merged [B] and [C] under the following treatments: [1] none; [2] and [4], PC1 and PC2 exposure in the dark for 1 day, respectively; [3] and [5], [2] and [4] followed by exposure to red light for 30 min followed by 1-day incubation, respectively. Cellular disintegration ([A]-[3] and [A]-[5]) and loss of GFP fluorescence ([B],[D]-[3] and [B],[D]-[5]) of the promastigotes was observed, indicative of their photo-inactivation.

FIG. 6 shows Leishmania tropica EP41 fluorescent cells of both stages. Left panel: promastigotes; right panel: axenic amastigotes. A-D, Phase contrast; A′-D′: FITC fluorescence; A-A′, B-B′: GFP transfectants; C-C′, D-D′: CFSE-loaded cells.

FIGS. 7A-7F show the results of MTT and fluorescence assays showing PC dose-dependent photo-inactivation of Leishmania tropica promstigotes (FIGS. 7A-7C) and axenic amastigotes (FIGS. 7D-7F). FIGS. 7A and 7D: MTT assay; FIGS. 7B and 7E: fluorescence assay for GFP release from egfp-transfectants; FIGS. 7C and 7F: fluorescence assay for FITC release from CFSE-loaded cells. Horizontal axes: C, Controls without PC-exposure; PC1 and PC2, Cells exposed to 0.01-1.0 μM PC1 or PC2 overnight, respectively; Black and blank bars, Samples with and without red light treatment. Vertical axes: % of control for MTT and fluorescence reduction normalized against the no-treatment controls. PC dose-dependent inactivation of cells was pronounced after red light exposure, irrespective of the assays used.

FIG. 8 shows the effect of amino-phthalocyanines on the growth of Leishmania tropica under dark conditions. Horizontal axis: Days for sample collection after cultivation of promastigotes in the presence of 0-1 μM PCs. Vertical axis: % of no PC controls for cellular GFP fluorescence. Left panel: Less stringent dark conditions: daily samples withdrawn from batch cultures with and without PCs in aluminum foiled-wrapped culture vessels. Right panel: more stringent dark conditions: cultures with and without PCs were pre-aliquoted in culture tubes wrapped with aluminum foil pre-sorted for daily sample collection.

FIG. 9 shows J774 macrophages after being exposed to 1 μM PC1 (1^{st} row), 1 μM PC2 (2^{nd} row) and PC2 followed by illumination with red light (3^{rd} row). Cells were observed under phase contrast for localization and for PC fluorescence using Cy5 filter. These data demonstrate localization of PCs in perinuclear endosomes, as shown in merged images and the absence of any noticeable changes in the cellular integrity after light exposure under the experimental conditions used (last row).

FIGS. 10A-10B show the disparity of parasite vs. host sensitivity to PC-mediated photo-inactivation. Cell viability of J774 macrophages and both Leishmania stages are shown after exposure to increasing concentrations of PC1 (FIG. 10A) and PC2 (FIG. 10B). Squares, J774 cells; triangles, axenic mastigotes; circles, promastigotes. Open and closed symbols signify cells with and without red light exposure for 20 min, respectively.

FIGS. 11A-11C show J774 macrophage processing of green fluorescent protein (GFP) delivered by amino-phthalocyanine (PC)-photoinactivated Leishmania GFP-transfectants.

FIGS. 12A-12C show quantitation of PC-mediated photo-inactivation of GFP-Leishmania delivered by three different schemes to J774 macrophages for processing. ***, significant p-value of 0.001 between dark and light samples.

FIGS. 13A-13B show intra-phagolysosomal degradation of oxidatively photo-inactivated Leishmania to release GFP for processing and precipitation of PC into inactive aggregates.

### DETAILED DESCRIPTION

All publications, patents and patent applications cited herein are hereby expressly incorporated by reference for all purposes.

Before describing the present invention in detail, a number of terms will be defined. As used herein, the singular forms “a”, “an”, and “the” include plural referents unless the context clearly dictates otherwise. For example, reference to a “protein” means one or more proteins.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

The disclosure provides systems and methods for delivering protein or peptide agents into a target cell. The delivery system comprises a biological carrier comprising an infective trypanosomatid protozoan, such as an infective Leishmania, selected or engineered to infect the target cell. The trypanosomatid is photosensitized using a phthalocyanine compound so that it is killed upon exposure to light; furthermore, the trypanosomatid expresses, or is transgenically modified to express, the desired polypeptide in the carrier. After the carrier is introduced into the target cell, the system is irradiated with light, such as visible light or light of specific wavelength, which leads to the death of the carrier and the subsequent release of the expressed proteins or peptides within the carrier into the target cell.

In some embodiments, the infective trypanosomatid is an infective Leishmania sp., which can include guinea pig Leishmania enriettii, rodent Leishmania turinica and reptilian Leishmania torentolae, and avirulent strains of pathogenic Leishmania spp., which are either laboratory-derived or genetically engineered by the strategies of molecular attenuation of virulence described.

In a preferred embodiment, the carrier is a non-pathogenic (but infective) trypanosomatid protozoan, such as members in the genera of Crithidia, Blastocrithidia, Herpetomonas, Phytomonas, Leptomonas, Trypanosoma and other non-pathogenic lower trypanosomatid protozoa.

Thus, in some embodiments, the target cell for the carrier is a mammalian cell. In some embodiments, the target cell is a mammalian macrophage. Other targets can include polymorphonuclear phagocytes, fibroblasts, and dendritic cells or any other cells susceptible to infection by genetically engineered Leishmania or other trypanosomatid protozoa with ligands specific to the targeted cells. Leishmania, for example, is known to specifically infect macrophages and dendritic cells. Leishmania cell- or tissue-specificity can be further genetically altered by incorporating ligand molecules from other cell-, tissue- or organ-specific parasites. Constructs so engineered are expected to “home-in” toward specific cells and tissues other than macrophages.

In some embodiments, genes of interest can be introduced into the carrier by incorporation into the chromosomal composition of the carrier. In some embodiments, genes can be introduced via standard plasmid vector techniques, or other techniques known in the art. In some embodiments, the gene of interest intended for introduction into the carrier can be one that serves any desired purpose, but preferably includes gene that expresses gene products that are therapeutic, prophylactic, and/or pharmacologically active. In some embodiments, the gene of interest can code for a product that will ultimately operate antigenically as a vaccine, such as a vaccine candidate.

The disclosure further provides methods of treating a mammal by delivering a protein or peptide to the mammal via the biological carrier described above. The biological carrier can be used to deliver the protein or peptide to a mammal to treat the mammal by administering the carrier encoding the protein or peptide of interest to the mammal. Administration of the carrier to a subject can be achieved by techniques that are well-known in the art. By way of example and not limitation, such administration techniques can include topical application, intramuscular injection, subcutaneous injection, intravenous administration and other parenteral, enteral, dermal routes of administration, inhalation, transbuccal, and nasal administration.

Genes encoding various peptides and proteins of potential prophylactic and/or therapeutic interest can be introduced into the trypanosomatid for expression and eventual release as a result of photosensitization and light exposure. Techniques for transfection of Leishmania with such genes are well known in the art. By way of example and not limitation, foreign genes of interest can be introduced into the chromosomal genome of the trypanosomatid, or they can be introduced by using well-known plasmid vector techniques. Examples of gene expression systems in these organisms are disclosed in International Patent Applications WO 02/44355 and WO 01/32896.

Other desirable genes can be introduced into the trypanosomatid for expression and ultimate release. The gene selected for introduction into the trypanosomatid will vary depending on the preferences of the practitioner of the present invention.

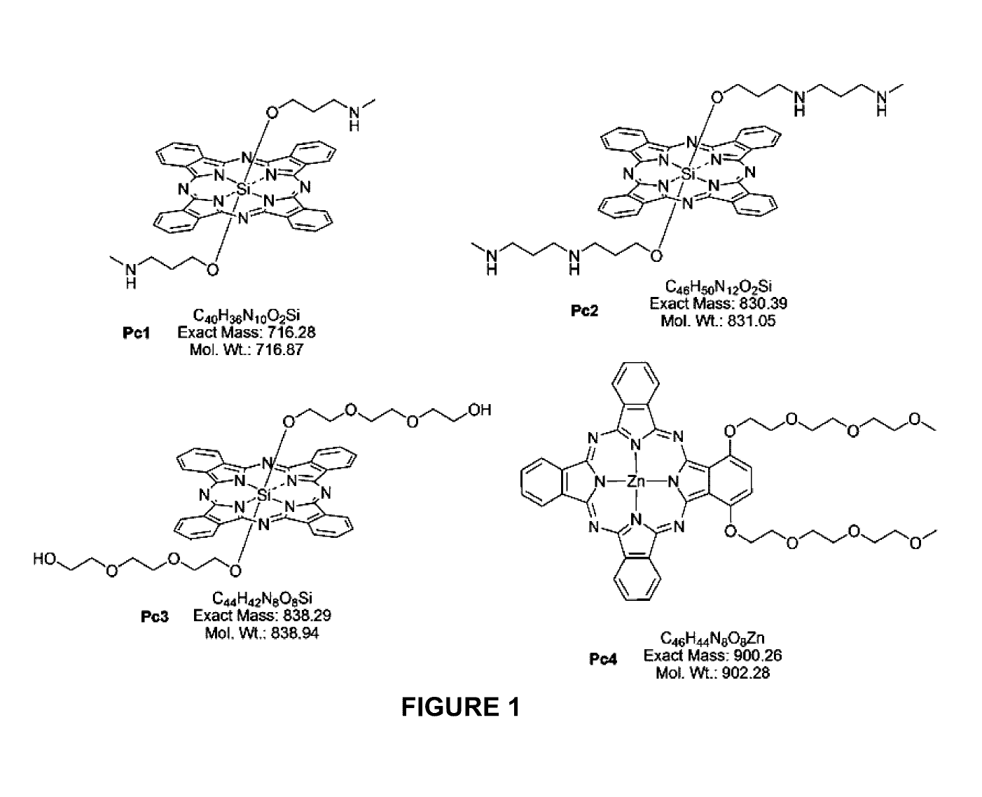

In embodiments where the trypanosomatid is Leishmania, the methods and compositions disclosed herein are contemplated for use in photodynamic vaccination by exploiting the unusual properties of Leishmania, i.e., the innate ability of Leishmania spp. to home to the phagolysosomes of antigen-presenting cells (the very cells which process and present antigenic epitopes to the mammalian immune systems for eliciting effective immunity) such as dendritic cells and macrophages. The vaccine candidate-expressing Leishmania constructs end up in the phagolysosomes of these antigen-presenting cells when delivered to animals as do the wildtype Leishmania. Light exposure of the photosensitized trypanosomatid lyses the intralysosomal Leishmania constructs, thereby concentrating vaccine candidate polypeptides for release and exposure to this antigen processing site for effective presentation. Leishmania has been reported to effectively deliver ovalbumin to macrophages for presentation of its antigenic epitopes to CD+ T-cells (Kaye et al., 1993, Eur. J. Immunol., 23:2311-2319). Time-controllable release of this and other antigens has not been incorporated into the previous schemes, but it is expected to heighten the level of the immune response. This is achievable through the use of photosensitized Leishmania according to the present disclosure. In addition, cytolysis of the photosensitized “carrier” Leishmania by visible light irradiation minimizes the risk of leishmaniasis, especially when non-pathogenic or avirulent species and strains are used. Both human and veterinary applications of the “vaccine” delivery by the Leishmania compositions of the disclosure can be considered under the same principles. The utility of this host-parasite model is contemplated for both in vitro and in vivo photodynamic therapy and vaccination by using exogenously supplied photosensitizers. Phthalocyanine (PC)-type photosensitizers of the disclosure contemplated for use in photodynamic therapy and photodynamic vaccination are shown in FIG. 1.

Thus, the methods and compositions of the disclosure are contemplated for use in vaccinating a human subject with a polypeptide (protein) vaccine candidate. The composition would include, in a preferred form of the invention, a Leishmania that expresses or has been transfected to express a cDNA sequence encoding the polypeptide vaccination candidate and one or more of the photosensitizers set forth herein loaded into the Leishmania for photo-inactivation to define a vehicle to deliver the protein vaccination candidate into a cell of a mammalian subject. Suitable routes of administration include oral, topical and transdermal. In some embodiments, it is also contemplated that the composition would specifically target mammalian macrophages or dendritic cells for presentation to T cells to cause an immune response.

Thus, in certain aspects of the disclosure, methods of delivering a polypeptide vaccination candidate to a cell of a mammalian subject includes the steps of: (1) providing a Leishmania that expresses a polypeptide vaccination candidate, such as a Leishmania transgenically modified to express a cDNA sequence encoding the protein vaccination candidate; (2) loading the Leishmania with an effective photosensitizer to sensitize the Leishmania as a carrier; (3) exposing the carrier to light to inactivate the Leishmania; and (4) delivering the carrier to the mammalian subject. In some embodiments, the Leishmania is exposed to or delivered to the mammalian subject prior to exposing the Leishmania to a photosensitizer, and prior to exposing the Leishmania to light. In some embodiments, the Leishmania is delivered or administered to the mammalian subject after exposure of the Leishmania to a photosensitizer, but prior to exposure of the Leishmania (and the mammalian subject) to light to deactivate the Leishmania.

The disclosure also relates to photodynamic therapy (PDT), in which photosensitizers (PS) that are light-excitable are used to produce, within a target cell or microorganism, cytotoxic reactive oxygen species (ROS) in the presence of atmospheric oxygen for clinical treatment of tumors, skin cancer and other cutaneous diseases. Photosensitizers can be applied exogenously, such is the case with phthalocyanines, or it can be induced endogenously with delta-aminolevulinate (ALA) to up-regulate heme biosynthesis of the target cells for over-producing photosensitive intermediates of this pathway, i.e. porphyrins. PDT has the potential to overcome the challenges of emerging drug-resistance in infectious and malignant diseases, since it is not known to elicit such resistance. Light and photosensitizers alone are non-cytotoxic and thus select no resistance phenotype. In combination, a burst of powerful cytotoxic ROS is rapidly generated, which simultaneously attack multiple cellular molecules, making it unlikely to induce the development of resistance.

In certain aspects, the present disclosure concerns the use of PDT for treatment of infectious diseases caused by trypanosomatid protozoa, for example, in the genus of Leishmania that cause leishmaniasis. Disclosed herein are phthalocyanine compounds that are significantly more effective (e.g., Leishmania are ˜50× more sensitive to PDT mediated by PC1/PC2 than PDT mediated by previously known phthalocyanines (Dutta et al, 2011, PLoS One 6:e20786)) and discriminating (e.g., EC50s of PC1/PC2-mediated PDT are in the nanomolar range for Leishmania, yet >10 μM for their host cells) than phthalocyanines previously identified for PDT of Leishmania.

Thus, in one aspect, the disclosure provides compositions for delivering a polypeptide into a mammalian cell, the composition comprising a trypanosomatid that has been treated with a compound of formula (I):

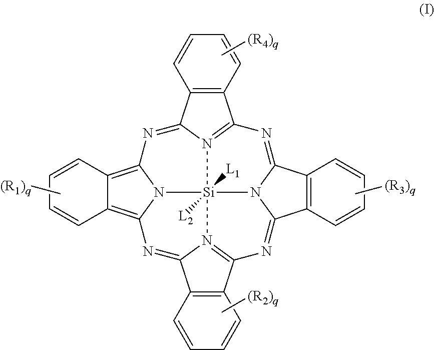

or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
   each R is independently —[C₁-C₆ alkylene-O]ₘ—R′ or —[C₁-C₆ alkylene-NR″]ₙ—R′,
   each m and n are independently an integer selected from 1 to 20,
   each R′ is independently selected from H and C₁-C₆ alkyl,
   each R″ is independently selected from H and C₁-C₆ alkyl;
   each q is independently an integer selected from 0, 1, and 2; and
   R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂;

wherein the trypanosomatid expresses the polypeptide.

In some embodiments, the trypanosomatid has been transgenically modified to express a cDNA sequence encoding the polypeptide. In some embodiments, the polypeptide is a vaccine candidate. In some embodiments, the trypanosomatid is capable of entering the mammalian cell. In some embodiments, the trypanosomatid has been exposed to light to inactivate the trypanosomatid following treatment of the trypanosomatid with the compound of formula (I).

As used herein, the terms “irradiate” and “expose to light” are interchangeable and refer to any means of exposing a cell, subject, or system to light, such as visible light, UV light, and infrared light. Typically, in the aspects and embodiments disclosed herein, the light is visible light. In some embodiments, the light is white light or sun light, inclusive of all effective wavelengths for phthalocyanines. In some embodiments, the light is red light. In some embodiments, the wavelength of the light is between about 600 and 700 nm. In some embodiments, the wavelength of the light is about 650 nm.

In some embodiments, the trypanosomatid is a Leishmania sp. trypanosomatid. In some embodiments, the Leishmania sp. is L. aethiopica, L. amazonensis, L. arabica, L. archibaldi, L. aristedesi, L. (Viannia) braziliensis, L. chagasi (syn. L. infantum), L. (Viannia) colombiensis, L. deanei, L. donovani, L. enriettii, L. equatorensis, L. forattinii, L. garnhami, L. gerbili, L. (Viannia) guyanensis, L. herreri, L. hertigi, L. infantum, L. killicki, L. (Viannia) lainsoni, L. major, L. mexicana, L. (Viannia) naiffi, L. (Viannia) panamensis, L. (Viannia) peruviana, L. (Viannia) pifanoi, L. (Viannia) shawi, L. tarentolae, L. tropica, L. turanica, or L. venezuelensis. In some embodiments, the Leishmania sp. Leishmania enriettii, Leishmania turinica, Leishmania torentolae, Leishmania tropica, or an avirulent strain of pathogenic Leishmania spp., either laboratory-derived or genetically engineered by known strategies of molecular attenuation of virulence. In some embodiments, the trypanosomatid is a non-pathogenic trypanosomatid protozoan, such as members in the genera of Crithidia, Blastocrithidia, Herpetomonas, Phytomonas, Leptomonas, Trypanosoma and other non-pathogenic lower trypanosomatid protozoa.

In some embodiments, the mammalian cell is a mammalian immune cell. In some embodiments, the mammalian immune cell is an antigen-presenting cell. In some embodiments, the mammalian immune cell is a dendritic cell or a macrophage or a B cell.

In another aspect, the disclosure provides methods for delivering a polypeptide to a cell of a mammalian subject, the methods comprising: (a) providing a trypanosomatid that expresses the polypeptide; (b) contacting the cell with the trypanosomatid by administering the trypanosomatid to the mammalian subject; (c) administering to the subject a photosensitizer, wherein the photosensitizer is a compound of formula (I):

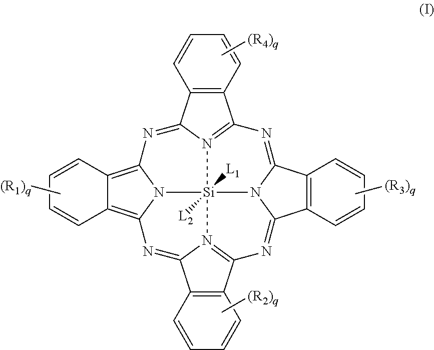

or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
   each R is independently —[C₁-C₆ alkylene-O]ₘ—R′ or —[C₁-C₆ alkylene-NR″]ₙ—R′,
   each m and n are independently an integer selected from 1 to 20,
   each R′ is independently selected from H and C₁-C₆ alkyl,
   each R″ is independently selected from H and C₁-C₆ alkyl;
   each q is independently an integer selected from 0, 1, and 2; and
   R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂; and

(d) exposing the subject to light to inactivate the trypanosomatid.

In another aspect, the disclosure provides methods for delivering a polypeptide to a cell of a mammalian subject, the methods comprising: (a) providing a trypanosomatid that expresses the polypeptide; (b) treating the trypanosomatid with a photosensitizer to produce a carrier, wherein the photosensitizer is a compound of formula (I):

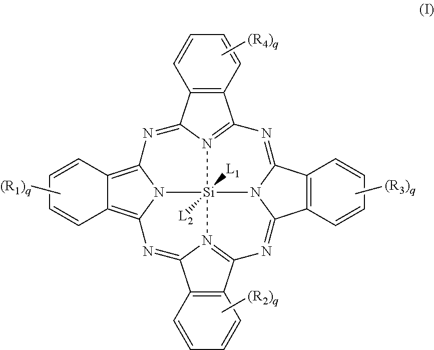

or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
   each R is independently —[C₁-C₆ alkylene-O]ₘ—R′ or —[C₁-C₆ alkylene-NR″]ₙ—R′,
   each m and n are independently an integer selected from 1 to 20,
   each R′ is independently selected from H and C₁-C₆ alkyl,
   each R″ is independently selected from H and C₁-C₆ alkyl;
   each q is independently an integer selected from 0, 1, and 2; and
   R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂;
      (c) contacting the cell with the carrier by administering the carrier to the mammalian subject; and
      (d) exposing the subject to light to inactivate the carrier.

In another aspect, the disclosure provides methods for delivering a polypeptide to a cell of a mammalian subject, the methods comprising: (a) providing a trypanosomatid that expresses the polypeptide; (b) treating the trypanosomatid with a photosensitizer to produce a carrier, wherein the photosensitizer is a compound of formula (I):

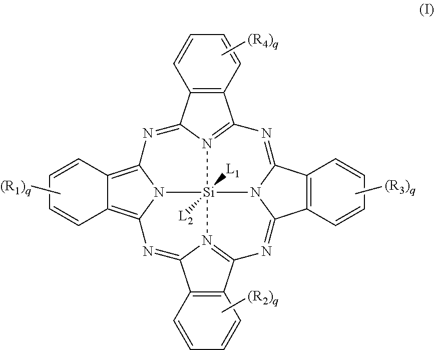

or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
   each R is independently —[C₁-C₆ alkylene-O]ₘ—R′ or —[C₁-C₆ alkylene-NR″]ₙ—R′,
   each m and n are independently an integer selected from 1 to 20,
   each R′ is independently selected from H and C₁-C₆ alkyl,
   each R″ is independently selected from H and C₁-C₆ alkyl;
   each q is independently an integer selected from 0, 1, and 2; and
   R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂;
      (c) exposing the carrier to light to inactivate the carrier; and (d) contacting the cell with the inactivated carrier by administering the carrier to the mammalian subject.

In some embodiments of the above methods, the trypanosomatid has been transgenically modified to express a cDNA sequence encoding the polypeptide. In some embodiments, the polypeptide is a vaccine candidate.

In some embodiments, the wavelength of the light is between about 600 and 700 nm. In some embodiments, the wavelength of the light is more than about 650 nm. In some embodiments, the trypanosomatid is a Leishmania sp. trypanosomatid. In some embodiments, the cell is an immune cell. In some embodiments, the immune cell is an antigen-presenting cell. In some embodiments, the immune cell is a dendritic cell or a macrophage.

In another aspect, the disclosure provides methods of treating a trypanosomatid infection and/or an infectious or malignant disease associated with a trypanosomatid infection in a subject, the method comprising administering to the subject a therapeutically effective amount of a compound of formula (I):

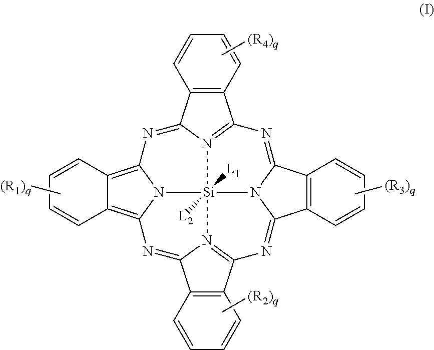

or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
   each R is independently —[C₁-C₆ alkylene-O]ₘ—R′ or —[C₁-C₆ alkylene-NR″]ₙ—R′,
   each m and n are independently an integer selected from 1 to 20,
   each R′ is independently selected from H and C₁-C₆ alkyl,
   each R″ is independently selected from H and C₁-C₆ alkyl;
   each q is independently an integer selected from 0, 1, and 2; and
   R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂.

In some embodiments, the methods further comprise exposing the subject to light following administration of the compound of formula (I). In some embodiments, the wavelength of the light is between about 600 and 700 nm. In some embodiments, the wavelength of the light is more than about 650 nm.

In some embodiments, the compound of formula (I) is administered to the subject topically, intramuscularly, subcutaneously, intravenously, parenterally, enterally, dermally, transbuccally, or intranasally. In some embodiments, the infection is a cutaneous, mucocutaneous, or visceral infection.

In some embodiments, the compound of formula (I) is administered to the subject in an in vitro vaccination procedure by: (a) treating antigen-presenting cells from the subject with the compound of formula (I) in vitro; and (b) administering the treated antigen-presenting cells to the subject. As used herein, the terms “in vitro vaccination” and “in vitro immunization” are interchangeable and refer to a process of isolating, and, optionally, culturing, antigen presenting cells, such as dendritic cells, from a subject. The isolated cells are then treated with or exposed to a vaccine candidate, following by re-introduction of the treated cells back into the subject. Alternatively, the treated cells are used to activate T-cells from the same subject. The activated T-cells are then returned to the subject for immunotherapy or cell therapy.

In some embodiments, the compound of formula (I) is administered at a dosage suitable for treating the trypanosomatid infection and/or the infectious or malignant diseases associated with the trypanosomatid infection. In some embodiments, the compound of formula (I) is administered at a dosage suitable to or sufficient to induce an immune response against Leishmania in the subject. In some embodiments, such as embodiments relating to in vitro vaccination, the compound of formula (I) is provided to antigen-presenting cells in vitro in an amount sufficient to induce an immune response against Leishmania, other infective agents and malignancy after the antigen-presenting cells are re-introduced into the subject. In some embodiments, the dosage of the compound of formula (I) ranges from about 0.1 mg/kg to about 10 mg/kg, such as from about 0.5 mg/kg to about 8 mg/kg, such as from about 1 mg/kg to about 5 mg/kg, such as about 3 mg/kg.

In some embodiments, the trypanosomatid infection is a Leishmania infection. In some embodiments, the Leishmania infection is caused by L. aethiopica, L. amazonensis, L. arabica, L. archibaldi, L. aristedesi, L. (Viannia) braziliensis, L. chagasi (syn. L. infantum), L. (Viannia) colombiensis, L. deanei, L. donovani, L. enriettii, L. equatorensis, L. forattinii, L. garnhami, L. gerbili, L. (Viannia) guyanensis, L. herreri, L. hertigi, L. infantum, L. killicki, L. (Viannia) lainsoni, L. major, L. mexicana, L. (Viannia) naiffi, L. (Viannia) panamensis, L. (Viannia) peruviana, L. (Viannia) pifanoi, L. (Viannia) shawi, L. tarentolae, L. tropica, L. turanica, or L. venezuelensis. In some embodiments, the Leishmania infection is an infection by Leishmania enriettii, Leishmania turinica, Leishmania torentolae, Leishmania tropica, or an avirulent strain of pathogenic Leishmania spp., either laboratory-derived or genetically engineered by known strategies of molecular attenuation of virulence. In some embodiments, the carrier is a non-pathogenic trypanosomatid protozoan, such as members in the genera of Crithidia, Blastocrithidia, Herpetomonas, Phytomonas, Leptomonas, Trypanosoma and other non-pathogenic lower trypanosomatid protozoa.

The term “therapeutically effective amount” refers to an amount administered to a subject that is sufficient to cause a desired effect in the subject.

The term “pharmaceutical formulation” or “pharmaceutical composition” refers to a preparation which is in such form as to permit a biological activity of an active ingredient to be effective, and which contains no additional components which are unacceptably toxic to a subject to which the formulation would be administered.

In some embodiments, the active ingredients of the compositions and methods disclosed herein are formulated as a pharmaceutically acceptable salt. As used herein, the term “pharmaceutically acceptable salt” refers to salts of the compounds of the present invention derived from the combination of such compounds and a pharmaceutically acceptable organic or inorganic acid (acid addition salts) or a pharmaceutically acceptable organic or inorganic base (base addition salts) which retain the biological effectiveness and properties of the compounds of the present invention and which are not biologically or otherwise undesirable. Examples of pharmaceutically acceptable salts include but not limited to those described in for example: “Handbook of Pharmaceutical Salts, Properties, Selection, and Use”, P. Heinrich Stahl and Camille G. Wermuth (Eds.), Published by VHCA (Switzerland) and Wiley-VCH (FRG), 2002. The compounds of the present invention may be used in either the free base or salt forms, with both forms being considered as being within the scope of the present invention.

In another aspect, the disclosure provides methods of deactivating a trypanosomatid, the methods comprising: (a) treating a live trypanosomatid with a compound of formula (I):

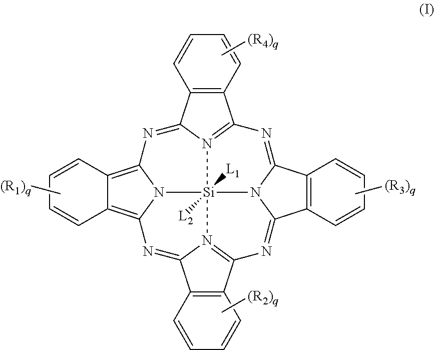

or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
   each R is independently —[C₁-C₆ alkylene-O]ₘ—R′ or —[C₁-C₆ alkylene-NR″]ₙ—R′,
   each m and n are independently an integer selected from 1 to 20,
   each R′ is independently selected from H and C₁-C₆ alkyl,
   each R″ is independently selected from H and C₁-C₆ alkyl;
   each q is independently an integer selected from 0, 1, and 2; and
   R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂; and
      (b) exposing the treated trypanosomatid to light in the presence of normally present atmospheric oxygen to deactivate the trypanosomatid.

In some embodiments, the light is white light. In some embodiments, the light is red light. In some embodiments, the wavelength of the light is between about 600 and 700 nm. In some embodiments, the wavelength of the light is more than about 650 nm. In some embodiments, the trypanosomatid is Leishmania.

In some embodiments, the compound of formula (I) is administered at a dosage suitable to deactivate the trypanosomatid.

In another aspect, the disclosure provides vaccine compositions comprising a deactivated trypanosomatid prepared by the above methods and a suitable pharmaceutical carrier.

The pharmaceutical or vaccine compositions of the disclosure can be administered to an animal, including human, by a number of methods known in the art. Examples of suitable methods include: (1) intramuscular, intradermal, intraepidermal, intravenous, intraarterial, subcutaneous, or intraperitoneal administration, (2) oral administration, and (3) topical application (such as ocular, intranasal, and intravaginal application).

Although an advantage of the vaccine methods and compositions disclosed herein is that they render the use of adjuvants unnecessary, in some embodiments, the vaccine methods compositions of the disclosure, e.g. Leishmania killed by oxidative photo-inactivation may optionally be used together with one or more adjuvants. Examples of suitable adjuvants include: (1) oil-in-water emulsion formulations (with or without other specific immunostimulating agents such as muramyl polypeptides or bacterial cell wall components), such as MF59™ (containing 5% Squalene, 0.5% Tween 80, and 0.5% sorbitan trioleate) and SAF (containing 10% Squalene, 0.4% Tween 80, 5% pluronic-blocked polymer L121, and thr-MDP); (2) saponin adjuvants, such as QS21, STIMULON™ (Cambridge Bioscience, Worcester, Mass.), ABISCO™ (Isconova, Sweden), or ISCOMATRIX™ (Commonwealth Serum Laboratories, Australia); (3) complete Freund's Adjuvant (CFA) and incomplete Freund's Adjuvant (IFA); (4) oligonucleotides comprising CpG motifs, i.e. containing at least one CG dinucleotide, where the cytosine is unmethylated; and (5) metal salt including aluminum salts (such as alum, aluminum phosphate, aluminum hydroxide); (12) a saponin and an oil-in-water emulsion.

In another aspect, the disclosure provides methods for inducing an immune response against a trypanosomatid in a mammalian subject comprising administering to the subject a vaccine composition as disclosed herein. In another aspect, the disclosure provides methods for inducing an immune response against Leishmania in a subject comprising administering to the subject a vaccine composition as disclosed herein, wherein the trypanosomatid is Leishmania. In some embodiments, the vaccine composition is administered to the subject in an amount effective to induce an immune response against the trypanosomatid, such as Leishmania.

In some embodiments of any of the methods and/or compositions disclosed herein, each q in formula (I) is an integer selected from 0 and 1. In some embodiments, each q in formula (I) is 0, as in the following formula:

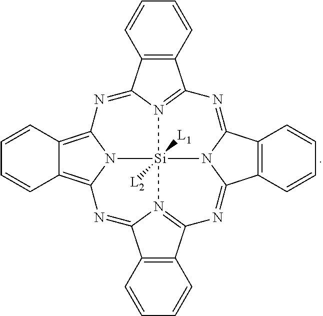

In some embodiments, L₁ and L₂ are independently selected from —O(C₁-C₆ alkyl), —O(C₁-C₆ alkenyl), —O(C₁-C₆ alkynyl), and —OR. In some embodiments, L₁ and L₂ are independently selected from —O(C₁-C₆ alkyl) and —OR. In some embodiments, L₁ and L₂ are independently —OR. In some embodiments, each R is independently —[C₁-C₆ alkylene-NR″]ₙ—R′, or -[ethylene-NR″]ₙ—R′, or -[propylene-NR″]ₙ—R′. In some embodiments, n is an integer selected from 1 to 6, or from 1 to 5, or from 1 to 4, or from 1 to 3, or n is 1, 2, or 3, or n is 1, or n is 2, or n is 3. In some embodiments, R″ is hydrogen or methyl, or R″ is hydrogen. In some embodiments, R′ is hydrogen or methyl, or R′ is hydrogen, or R′ is methyl. In some embodiments, L₁ and L₂ are independently selected from:

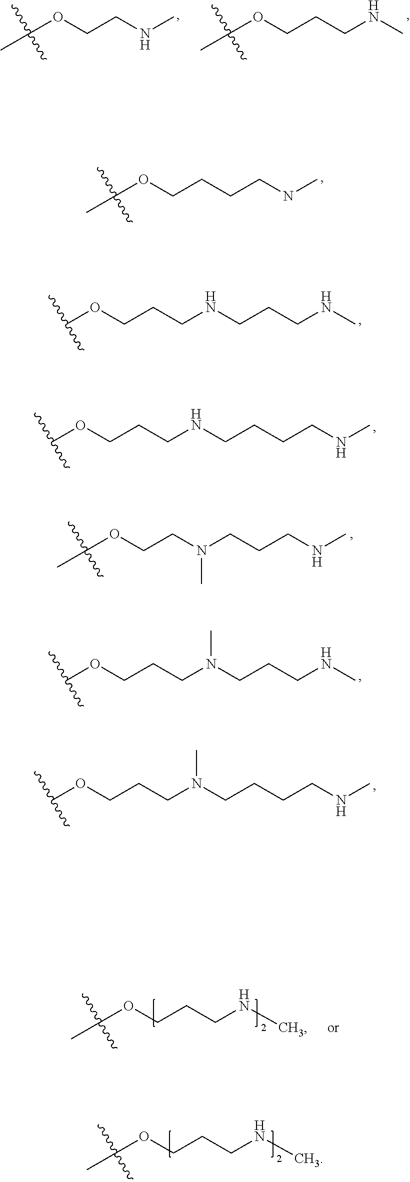

In some embodiments, L₁ and L₂ are independently selected from:

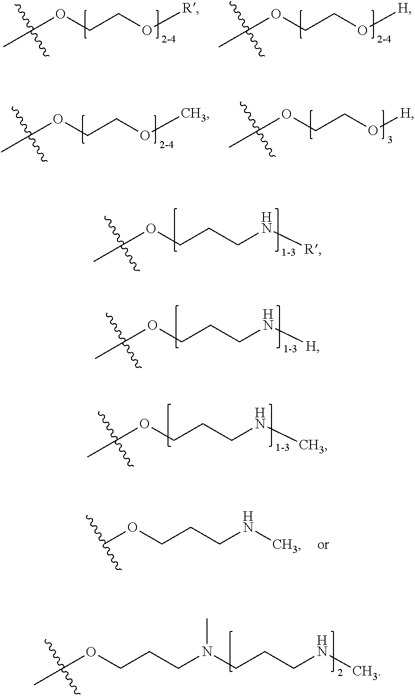

In some embodiments, L₁ and L₂ are independently selected from:

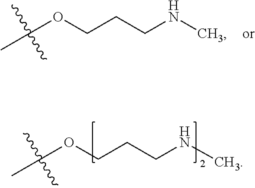

In some embodiments, L₁ and L₂ are the same. In some embodiments, the compound of formula (I) is:

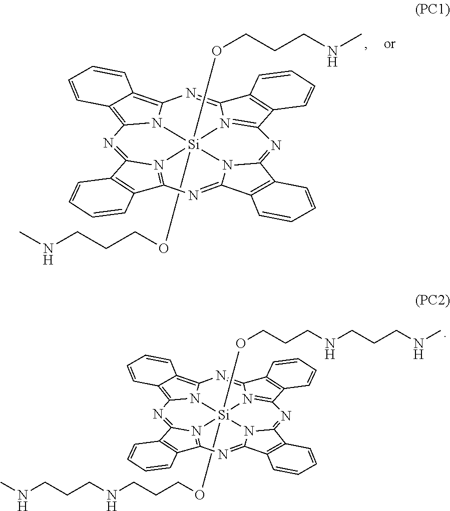

### EXAMPLES

The Examples that follow are illustrative of specific embodiments of the invention, and various uses thereof. They are set forth for explanatory purposes only, and are not to be taken as limiting the invention.

### Example 1

### Methods for Identification of Novel Phthalocyanines (PCs) for PDT of Leishmania

Novel PCs were screened, leading to the identification of amino-PC conjugates (PCs 1 and 2), which are significantly more effective and discriminating than PCs previously identified for photodynamic inactivation of Leishmania. This was demonstrated in both stages of L. tropica qualitatively by fluorescent microscopy and fluorimetry for the uptake of PCs 1 and 2, and quantitatively by fluorescence/metabolic cell viability assays after photo-inactivation. The effectiveness and discriminatory potential of PCs 1 and 2 are predicted from the large disparity between Leishmania and J774 macrophages in their EC₅₀ values for photo-inactivation.

Phthalocyanines.

Four phthalocyanines (FIG. 1) were synthesized and tested: amino-phthalocyanines PC1 and PC2; triethylene glycol substituted Zn(II)-phthalocyanine, PC4; and PC3, which was prepared by treating silicon (IV) phthalocyanine dichloride with triethylene glycol in toluene and purified by silica gel column chromatography (see Jiang et al., 2011, J Med Chem. 54: 320-330; and Liu et al., 2009, Dalton Trans. 2009: 4129-4135). For PC3: ¹H NMR (300 MHz, CDCl₃): δ 9.65-9.68 (m, 8H, Pc-H), 8.34-8.37 (m, 8H, Pc-H), 3.31-3.36 (m, 4H, OCH₂), 2.94-2.97 (m, 4H, OCH₂), 2.41-2.44 (m, 4H, OCH₂), 1.49-1.52 (m, 4H, OCH₂), 0.49 (t, J=5.1 Hz, 4H, OCH₂), −1.90 (t, J=5.1 Hz, 4H, OCH₂). ¹³C{¹H} NMR (75.5 MHz, CDCl₃): δ 149.3, 136.0, 130.9, 123.7, 71.8, 69.4, 69.3, 68.5, 61.4, 55.0. MS (ESI): m/z 861 [M+Na]⁺ (100%). HRMS (ESI): m/z calcd for C₄₄H₄₂N₈NaO₈Si [M+Na]⁺, 861.2787. found 861.2797. Anal. Calcd for C₄₄H₄₂N₈O₈Si: C, 62.99; H, 5.05; N, 13.36. Found: C, 62.43; H, 5.04; N, 13.08.

All compounds were dissolved in a suitable solvent, such as dimethyl sulfoxide (DMSO), for preparing 1 mM stock solutions, from which working solutions (0.01 to 10 μM) were prepared by serial 10-fold dilutions.

Cells.

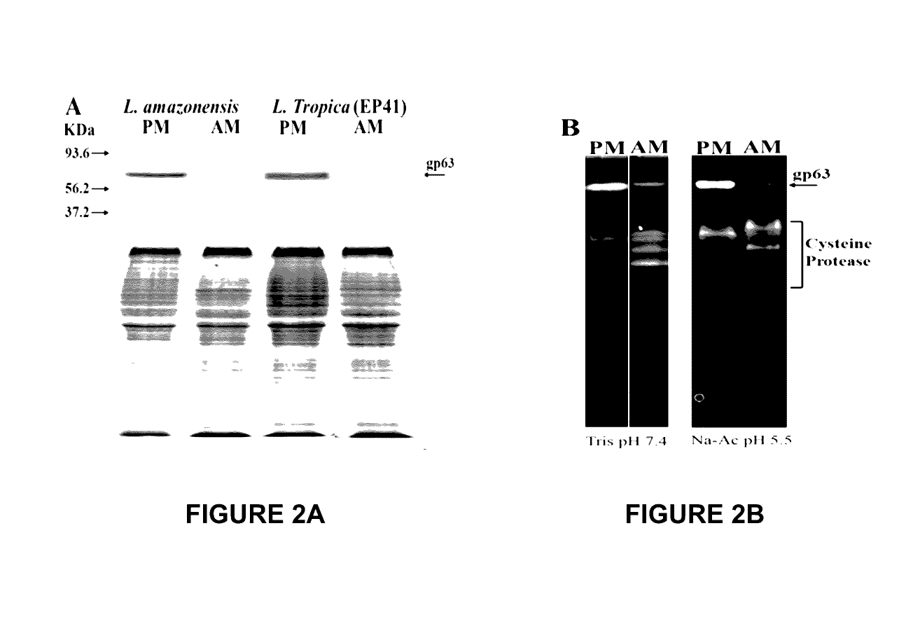

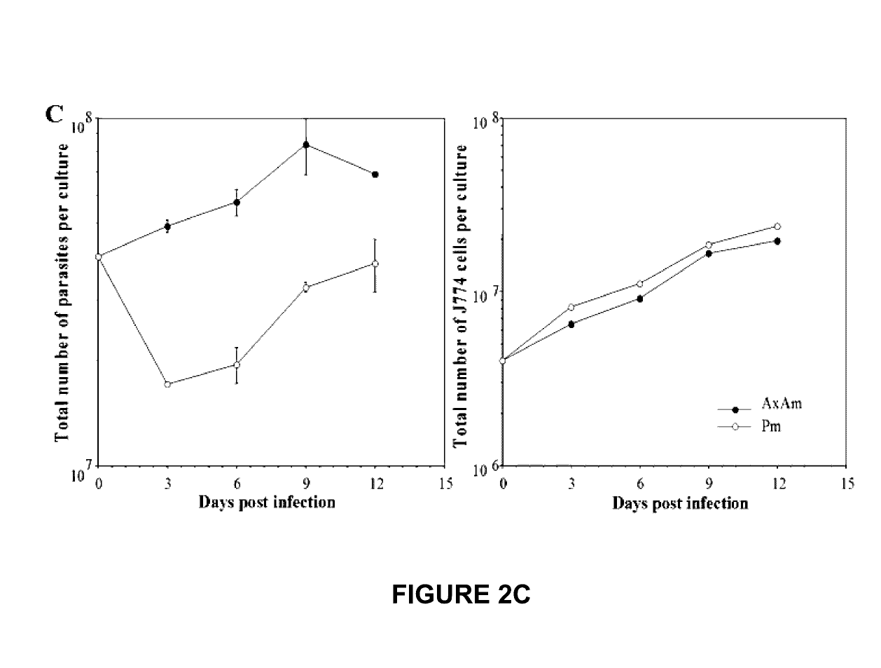

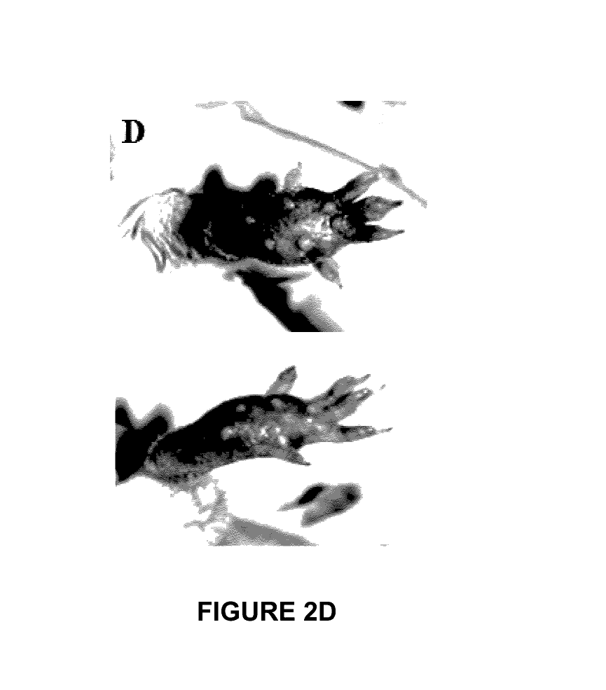

Leishmania used included 2 virulent strains of L. tropica EP41 (FIG. 2) and CBU10. EP41 was chosen on the basis of: (1) excellent growth of EP41 as promastigotes (Pm), reaching a cell density of approx. 50×10′ cells/ml in 3-4 days; and (2) successful differentiation of EP41 Pm into axemic amastigotes (AxAm) at 33 C, pH 5.3 and their stability for continuous cultivation as such. This differentiation was verified as authentic not only morphologically but also by molecular and biochemical characterizations of stage-specific proteases.

Promastigotes were grown to stationary phase at 25° C. in HEPES-buffered Medium 199 or Schneider's Medium with 10% heat inactivated fetal bovine serum (HIFBS) and as axenic amastigotes at 33° C. in Schneider's Medium with 10% HIFBS, pH 5.3. Macrophages of the J774 cell line were cultured in RPMI-1640 medium plus 10% HIFBS. J774 macrophages were infected at 35° C. with both stages grown to stationary phase at a ratio of 1:10 (4×10⁶ macrophages/40×10⁶ Leishmania/4 ml RPMI+10% HIFBS in a 25 cm²TC flask). The rate of infection was quantitatively determined at the time points indicated by microscopic counting of ˜100 cells for % infection and the average # of Leishmania/cell. The total # of Leishmania per culture determined from these data are shown in FIG. 2C, left panel; the total # of J774 macrophages/culture are shown in FIG. 2C, right panel. AxAm were observed to be more infective than Pm.

Footpad lesions were also observed on BALB/c mice after L. tropica infection (FIG. 2D). Intradermal infection with 10⁷ Leishmania for up to 45 days produced footpad swelling, which is more pronounced with AxAm than with Pm, as shown in the upper and lower panels of FIG. 2D.

GFP Transqenic Leishmania.

L. tropica EP41 and CBU10 were transfected by electroporation with egfp-p6.5 to express green fluorescent protein (GFP) (see Dutta et al., 2005, Antimicrob Agents Chemother 49: 4474-4484). The transfectants obtained were selected using 10 μg/mL tunicamycin.

CFSE Loading of Leishmania.

Leishmania were exposed for 10 min at 37° C. to 5-10 μM carboxyfluorescein succinimidyl ester (CFSE) (Invitrogen) at 10⁸ cells/mL in Hank's Balanced Salt Solution (HBSS) HEPES-buffered to pH 7.4 plus 0.01% bovine serum albumin (HBSS-BSA). CFSE was converted by intracellular non-specific esterase into cell-impervious fluorescent products, rendering live Leishmania fluorescent.

Photosensitization of Cells.

Leishmania were suspended to 5×10⁷-10⁸ cells ml⁻¹ in HBSS-BSA for exposure to serial dilutions of PCs 1-4 in the dark overnight. Controls included cells exposed to diluent alone. J774 macrophages were treated as monolayer in HBSS-BSA or culture medium under otherwise the same conditions. All cultures were kept in the dark for various time periods up to 24 hrs.

Exposure of Photo-Sensitized Cells to Red Light.

Control and PC-sensitized Leishmania were washed and suspended in HBSS-BSA. The cell suspensions in 24-well culture plates and monolayers of J774 macrophages in 25 cm² TC flasks were exposed from the bottom to red light (wavelengths >650 nm) via a red filter (Smith-Victor Co., Barlett, Ill., USA; part #650021) over a light box for 20-30 min at a fluence of 1-2 J cm².

MTT Reduction Assay.

An MTT reduction assay was performed according to manufacturer's protocol (Sigma, Molecular Probe kits). Freshly prepared MTT stock solution (5 mg ml⁻¹) was added to promastigote and axenic amastigote cell suspensions at 0.5-1.0×10⁸ per mL. Formazan formed were solubilized for reading in a Biotek Synergy HT plate reader (Texas Instruments) and analyzed by using GEN 5 version 5.11.1 software.

Quantitative Fluorescence Assay.

GFP transfectants or CFSE-loaded Leishmania in HBSS-BSA were dispensed in triplicate 100-μl aliquots at 5×10⁶ cells/well to 96-well microtiter plates for detergent lysis with 0.5% (v/v) Nanodet P-40 to release cellular GFP/CFSE. The lysates were read for fluorescence at a sensitivity of 60 in a BIO-TEK Synergy HT plate reader (excitation wavelength of 485/20 nm and emission wavelength of 528/20 nm). Data collected were analyzed by using GEN 5 version 5.11.1 software.

Leishmania Uptake of PCs Assessed by Fluorimetry.

Promastigotes were exposed in the dark to 0.1 μM PC1 and PC2 at 5×10⁷ cells ml⁻¹ under non-growing conditions in HBSS-BSA. Controls were set up without cells under the same conditions. Samples were dispensed in 1-ml aliquots in the dark at 25° C. and harvested at different intervals for up to 24 hrs. Cells were washed once and sedimented as pellets stored at −20° C. At completion of the experiments, 2.5 ml of DMSO were added to each frozen pellet for extraction of PC. The fluorescence intensities of the extracts were determined (excitation wave length at 608 nm, Emission wavelength at 682 nm) in a LS-50B spectrofluorimeter (Perkin-Elmer) using FL WINLAB software. Concentrations of the PC were expressed in nmol per 10⁷ cells by extrapolating their fluorescence intensities against those from a linear standard curve of PC concentrations from 0 to 20 nM.

Assays for Dark Toxicity of PCs.

GFP transfectants at 5×10⁶ ml⁻¹ in culture media were divided into 12-ml aliquots for exposure to a mixture of PC1 and PC2 in 10× serial dilutions from 0 to 1 μM. Cultures were handled in two ways to minimize light exposure: Normally used conditions: These were the conditions referred to as “in the dark” in all experiments described. Cells were exposed in 12-ml aliquots to 0 to 1 μM PCs each in individual culture vessels wrapped in aluminum foil. Culture vessels were unwrapped for daily sample collection under the incipient light with room light turned off. More stringent conditions: Samples from different experiments to be collected daily were aliquoted (3 ml) and wrapped together in aluminum foil for keeping them in total darkness for the duration of the incubation. Daily sample collection under both conditions took ˜10 min under incipient light. Cell pellets were frozen at −20° C. Frozen samples were processed for cellular GFP fluorescence assay as described above. Data were presented as % of the control values at each time point from untreated cells.

Fluorescence Microscopy.

Nikon Eclipse 80i and TE2000-S microscopes with CCD cameras were used with Metamorphosis (version 6.1) or NIS Elements version AR 4.20.01 software for image capture and analysis. Concentrated cells (˜3 μl) on a glass slide under an 18 mm² glass coverslip were first scanned under phase contrast to localize cells. Cells exposed to PC1, PC2, PC3, and PC4 were then imaged for subcellular localization of PC fluorescence by using Cy5 filter set. GFP/CFSE fluorescent Leishmania were further examined by using the FITC filter set. Images captured under different settings were merged by using the software programs mentioned. The filter sets (Chroma Technology Co., Brattleboro, Vt.) used were: (1) HQ620/60 (620-nm exciter), Q660LP (660-nm dichroic), and HQ700/80 (700-nm emitter) for phthalocyanines; and (2) HQ480/40 (480-nm exciter), Q505LP (505-nm dichroic), and HQ535/50 (535-nm emitter) for GFP and CFSE.

Data Analysis/Presentation.

In the Examples disclosed herein, all experiments were repeated at least twice and in most cases three times using mainly EP41 and CBU10 for confirmation. Results obtained were comparable among repeat experiments. Data presented represent mean±standard errors of the values in triplicate for each of individual samples from representative experiments.

### Example 2

### Leishmania Uptake of Amino-Phthalocyanine Conjugates (PCs 1 and 2)

Of four phthalocyanines (PCs 1-4) examined (FIG. 1), PC1 and PC2 were detected by fluorescent microscopy in Leishmania, which are axially substituted each with two symmetrical ligands, consisting of mono- and di-amino groups, respectively. There was no uptake of PC3 and PC4 by Leishmania, irrespective of the concentrations and periods of incubation used (not shown).

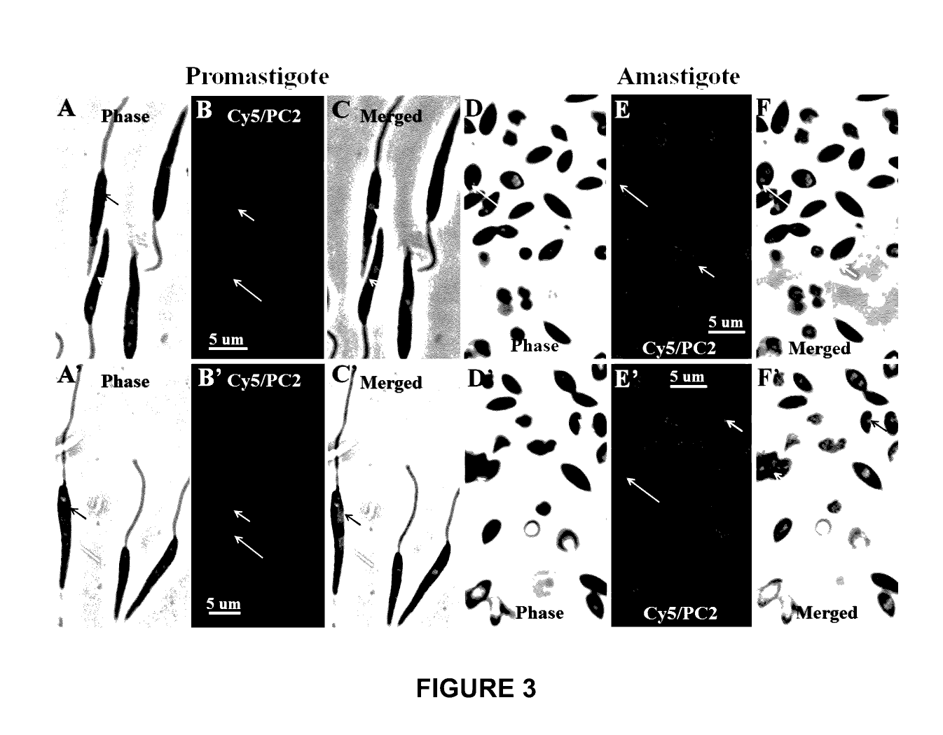

PCs 1 and 2 were taken up by both Leishmania stages, as noted in the images captured by using Cy5 filter set (results for PC2 are shown in FIG. 3; PC1 produced similar results to PC2, though results for PC1 are not shown). In FIG. 3, left panel: promastigotes A-C/A′-C′; right panel: axenic amastigotes D-F/D′-F′. A/A′, D/D′: phase contrast; B/B′, E/E′: Cy5 fluorescence for phthalocyanine PC2; C/C′, F/F′: merged images. Panels A-F and A′-F′ show different fields of view for the same treatment groups. The uptake of both PCs was time-dependent. Cells examined immediately after exposure to 0.1-1 μM PCs 1 and 2 showed no intracellular fluorescence. Fluorescence began to emerge in cells exposed to the PCs for ≧1 hour and increased gradually in intensity upon further incubation. After ≧4 hours, PC fluorescence was readily discernable in discrete cellular entities of promastigotes with their anterior flagellum as a reference structure for orientation (FIG. 3, promastigote). PC fluorescence appeared in vacuoles in the proximity of the flagellar pocket (short arrow), in multi-vesicular tubular structures and vacuoles (long arrow) distant from the flagellar pocket (FIG. 3, promastigotes A-C, A′-C′ merged images). The axenic amastigotes also appeared to have PC fluorescence in the similar intracellular structures, (FIG. 3, amastigote, D-F arrows), although there is no external flagellum to facilitate their identification in these small cells. PC fluorescence appeared to vary considerably with individual amastigotes (FIG. 3, amastigote D-F), but it was present in all when the fluorescence intensity was digitally optimized (amastigote D′-F′). Fluorescence intensity appeared higher in both stages after exposure to PC2 than to PC1.

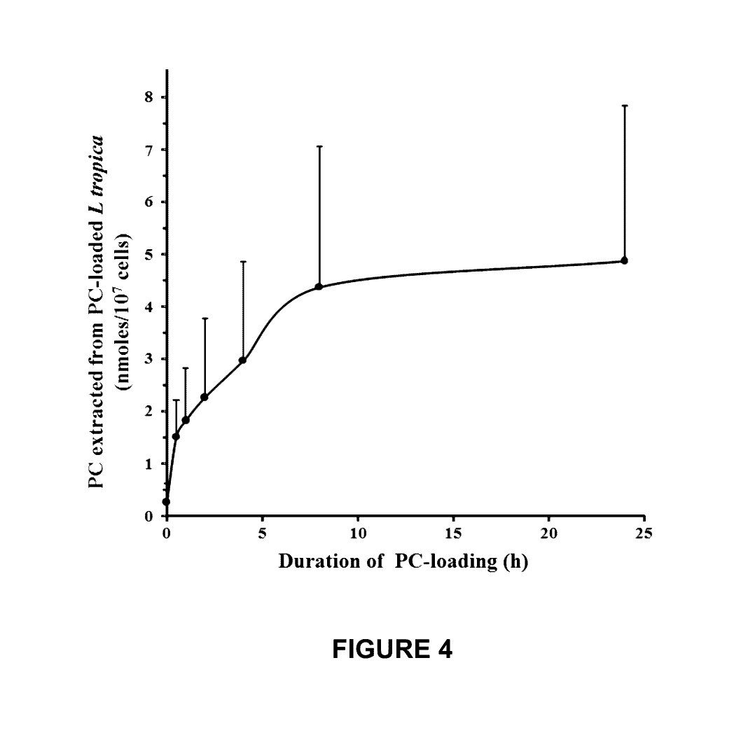

The uptake of PCs 1 and 2 followed saturation kinetics, as determined by fluorometric quantitation of cell-associated PC (FIG. 4). Since the cells were washed before PC extraction, cell surface associated PC is excluded from the readings obtained. Spontaneously precipitation of PCs and cell proliferation do not contribute to the readings, since both were found minimal within the timeframe and conditions of incubation. Thus, the rise of cell-associated PC with time is reflective of its uptake, reaching a plateau in ˜7 hrs. The kinetics of the uptake is comparable among different experiments, although the readings varied significantly from one experiment to another, as reflected in the large standard errors (FIG. 4). This was attributed to the inherent variations of different lots of cells used and/or different efficiencies of PC extraction from different sample sets.

### DISCUSSION

The uptake of PS by Leishmania is a prerequisite for its PDT effectiveness. PCs 1 and 2 are clearly taken up by both stages of L tropica, as evidenced by fluorescent microscopy (FIG. 3). The emergence of PC-positive vesicles near the flagellar pockets, lysosome-like multi-vesicular bodies and in distal vacuoles are all structural recapitulation of endocytic pathway, as shown previously by using different PSs of similar properties, including PCs 14 and 15. The uptake of all these PCs by Leishmania is thus akin to endocytosis of soluble molecules at the lining membrane of their flagellar pocket where endosomes are formed for shuttling endocytosed cargo to lysosomes. The saturation kinetics of the PC uptake observed is consistent with this interpretation (FIG. 4). The absence of PC fluorescence in the cytosol is indicative of very little, if any uptake of PCs 1 and 2 via transporters of the plasma membrane, e.g. polyamines, consistent with the previous finding that spermidine did not compete with PCs 1 and 2 for their uptake by mammalian cells (see Jiang et al., 2011, J Med Chem. 54: 320-330).

### Example 3

### PC1- and PC2-Mediated Sensitization of Leishmania

Microscopy of Leishmania Photolysis Mediated by PCs 1 and 2.

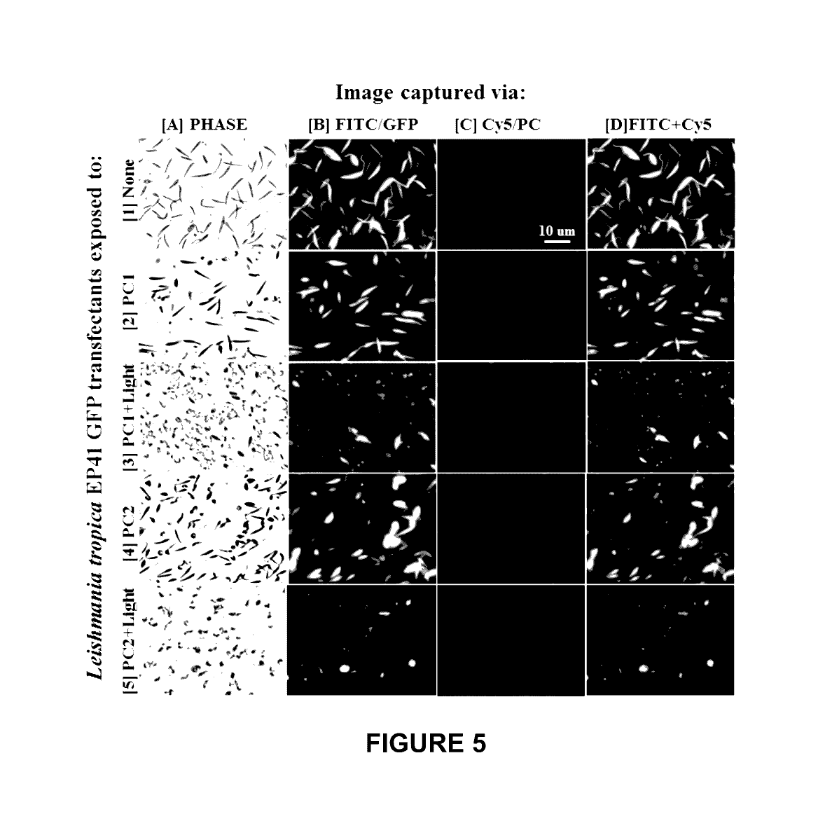

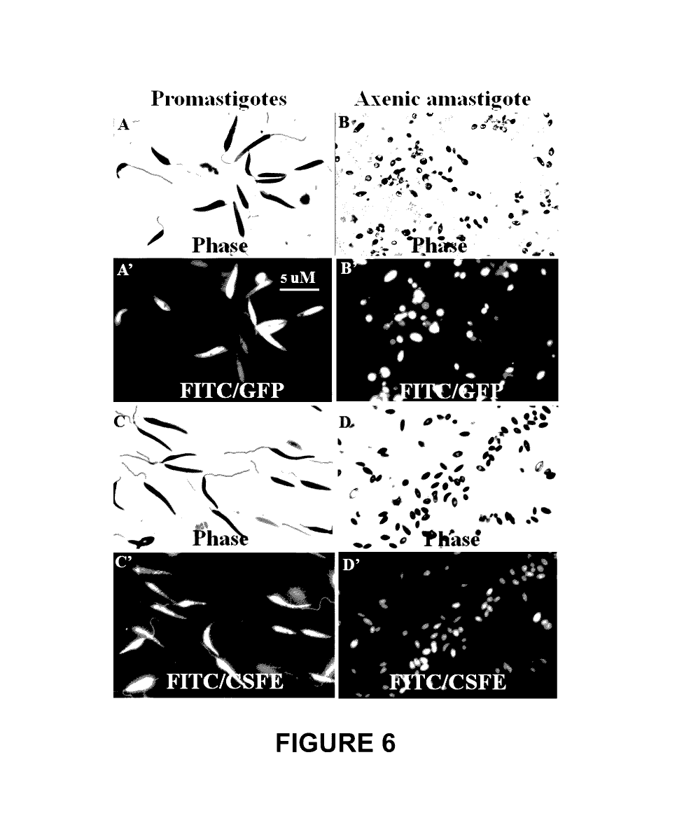

When sensitized with PCs 1 and 2 followed by exposure to red light, promastigotes were found to lose their motility (not shown) and structural integrity (FIG. 5, column [A], rows [3] and [5]). In contrast, they remained motile and intact in the groups of no treatment or treated with PCs 1 and 2 alone without red light exposure (FIG. 5, column [A], rows [1], [2], and [4]). This was further indicated by the loss of cellular fluorescence when using GFP/CFSE fluorescent cells (FIG. 6). After red light exposure of PC1- and PC2-loaded GFP transfectants, the GFP fluorescence was reduced, especially with PC2 (FIG. 5, columns [B] and [D], rows [3] and [5]), but not in the control groups of no treatment and PC1 and PC2 treatment alone (FIG. 5, columns [B] and [D], rows [1], [2], and [4]). The images taken under Cy5 filter showed that PC1 and PC2 fluorescence was absent in untreated cells (FIG. 5, columns [C] and [D], row [1]), as expected and differed little among PC1- and PC2-treated cells with or without red light exposure (FIG. 5, column [C], rows [2] through [5]). In merged images, PC-GFP co-localization was evident in cells treated with PC1 and PC2 alone (FIG. 5, column [D], rows [2] and [4]), but not at all or less so after exposure to red light (FIG. 5, column [D], rows [3] and [5]).

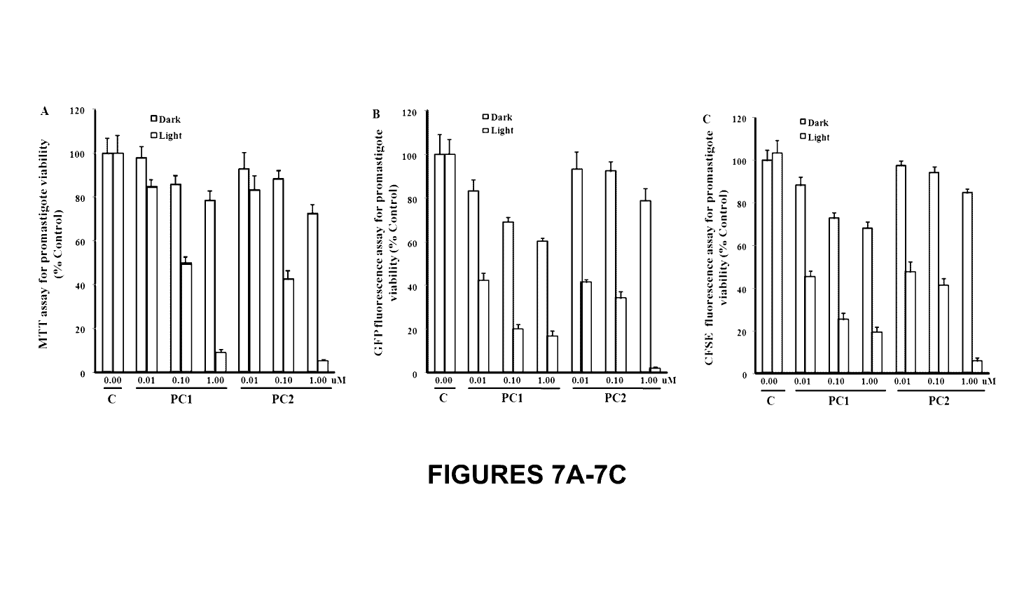

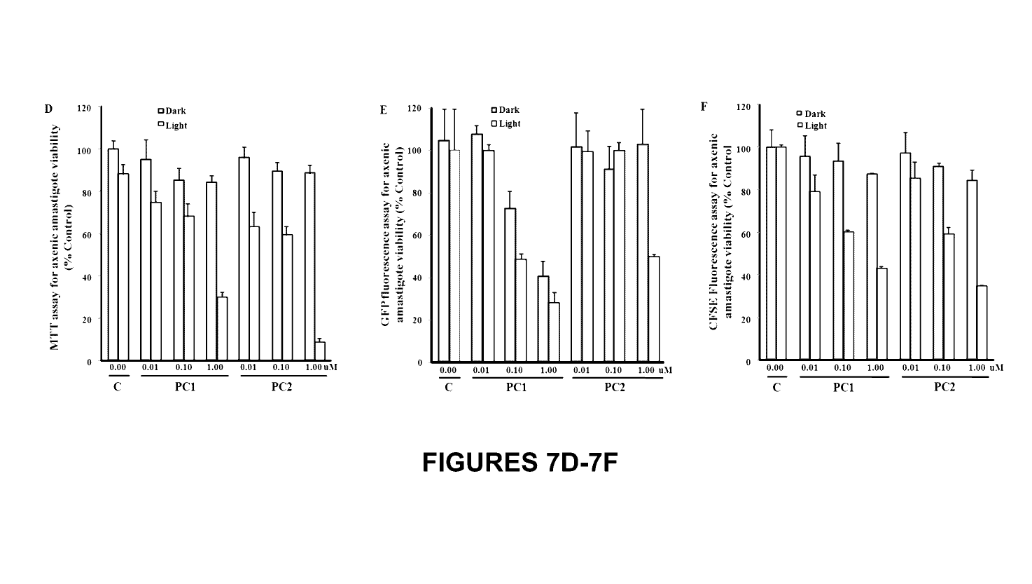

Similar results were obtained by using other fluorescent EP41 and CBU10 Leishmania cells (not shown, but see quantitative data presented in FIG. 7).

In the presence of PCs 3 and 4, Leishmania showed no cellular fluorescence, and retained their motility and cellular integrity with or without red light exposure (not shown).

PC Dose-Dependent Sensitization of Leishmania for Photo-Inactivation.

MTT reduction and loss of cellular GFP/CFSE fluorescence assays showed that both promastigotes (FIGS. 7A-7C) and axenic amastigotes (FIGS. 7D-7F) lost their viability progressively with exposure to increasing concentrations of PC1 and PC2 from 0 to 1 μM (FIGS. 7A-7F). The loss of fluorescence was significantly greater among red light-exposed samples (All blank bars) than those not exposed to red light (All dark bars). Promastigotes (FIGS. 7A-7C, blank vs dark bars) were seen to respond more consistently to both PC1 and PC2 than axenic amastigotes (FIGS. 7D-7F, blank vs dark bars). The differences between red light and no-light for each PC concentration used are significantly greater for promastigotes (FIGS. 7A-7C, blank vs dark bars) than axenic amastigotes (FIGS. 7D-7F, blank vs dark bars), indicating that the former are more susceptible to PC1- and PC2-mediated photo-inactivation. This is also evident by comparing each set of the values under the light condition at each PC concentration for both stages, especially at the highest concentration of PC2 (FIGS. 7A-7C vs. FIGS. 7D-7F, blank bars at PC 2, 1 μM). At the highest PC concentration of 1 μM used, PC2 appeared to be more effective than PC1 against promastigotes (FIGS. 7A-7C, PC1 vs PC2, blank bars at 1 μM). This was less pronounced for axenic amastigotes (FIGS. 7D-7F, PC1 and PC2, blank bars at 1 μM), especially PC1-treated GFP transfectants in response to red light (FIG. 7E, dark vs light bars at PC1 0.01-1.0 μM).

Growth of Promastigotes in the Presence of PC1 and PC2.

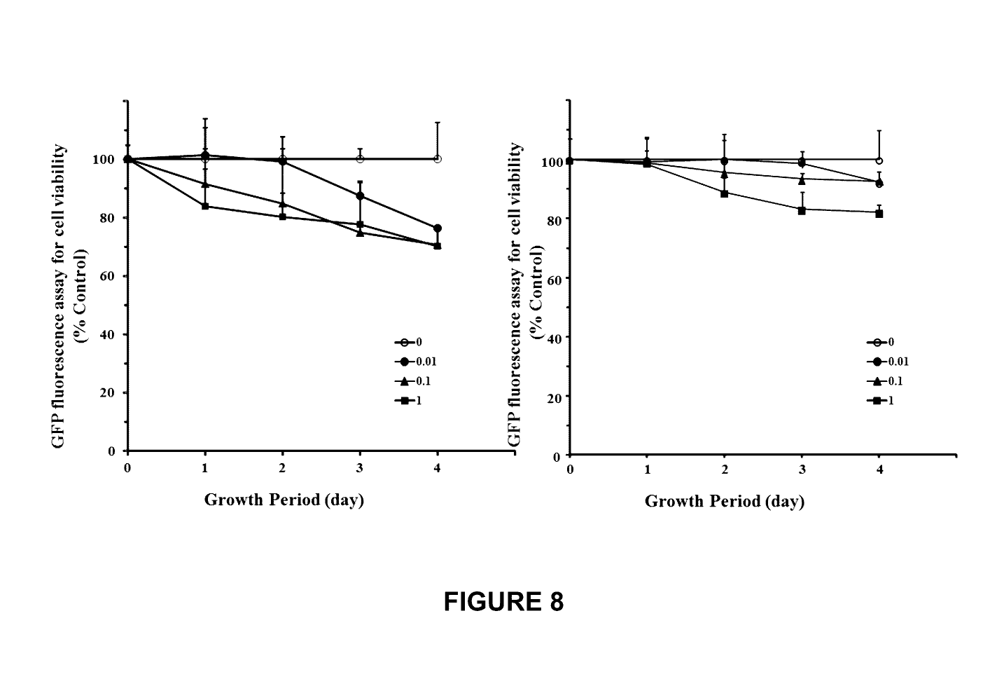

Room light exposure was minimized under normally used and more stringent conditions as described (see Materials and Methods). Daily collected samples included small aliquots from all for brief microscopic observations, which showed no gross differences in motility, integrity and density of the promastigotes exposed to all PC concentrations (0-1 μM) under both “dark” conditions throughout the incubation period of 4 days. GFP fluorescence assays of the samples showed differences in PC-mediated growth inhibition between the two “dark” conditions. Under normally used conditions, GFP fluorescence was reduced in samples exposed to all PC concentrations used by up to 25% of the PC-untreated controls on day 4 (FIG. 8, left panel). The loss of fluorescence increased progressively with time at PC concentrations from 0.1 to 1 μM, but was not evident until day 2 at 0.01 μM. These data (FIG. 8, left panel) are consistent with those for photo-inactivation under “dark” conditions presented in FIGS. 7A-7F. Under dark conditions controlled more stringently, there was also loss of GFP fluorescence at all PC concentrations used, but to a much smaller extent, especially notable at 0.01-0.1 μM (FIG. 8, right panel).

### DISCUSSION

These results demonstrate that PCs 1 and 2 taken up by L. tropica sensitize them effectively to red light for photo-inactivation. This is underscored by the facts that PCs 3-4 are not taken up and have no photodynamic activity, consistent with previous observations using similarly ineffective PCs. Qualitative and quantitative data provided convincing evidence for PC1- and PC2-mediated photo-inactivation of Leishmania. This is indicated clearly by seeing red light-induced cellular changes of PC-loaded Leishmania, i.e. flagellar immobilization, cell disintegration, and loss of cellular GFP/CFSE fluorescence (FIG. 5). This loss of cell viability as indicated is conclusively demonstrated quantitatively by three different assays, showing that it is PC dose-dependent for both stages after red light exposure (FIG. 7). The use of three different methods lends credence to the results obtained. Some discordance of the data obtained from the 3 different methods is not unexpected, considering that they are based on different mechanisms, i.e. MTT reduction activity and detergent release of fluorescence from GFP/CFSE labeled cells. While the MTT assay is a standard assay used extensively to assess cell viability, it is less sensitive than the fluorescence assays developed (data not shown). The two fluorescence assays have relative merits for assessing anti-Leishmania PDT. Cellular GFP is sensitive as a protein to oxidative photo-denaturation for loss of fluorescence, but its use is limited by the necessity of producing transfectants for each species. In contrast, CFSE is universally applicable to any Leishmania by one-step incubation, although the fluorescence is less detergent soluble, thereby reducing its sensitivity. Together, the qualitative and quantitative data presented show clearly that PC1 and PC2 are effective photosensitizers for anti-Leishmania PDT.

The efficacy of PC1- and PC2-mediated photo-inactivation of Leishmania is actually greater than what is reflected in the differences seen between dark and light conditions used (FIG. 7). Unavoidable incipient light contributes to photo-inactivation of PC-loaded Leishmania under the routine dark conditions used, since the growth inhibition under these conditions was reduced when dark conditions were controlled more stringently (FIG. 8). While dark toxicity of PCs 1 and 2 to Leishmania cannot be totally ruled out, it appears to be rather marginal in comparison to their anti-Leishmania photo-inactivation.

### Example 4

### Differential Sensitivity of Leishmania Versus J774 Macrophages to PC1- and PC2-Mediated Photo-Inactivation

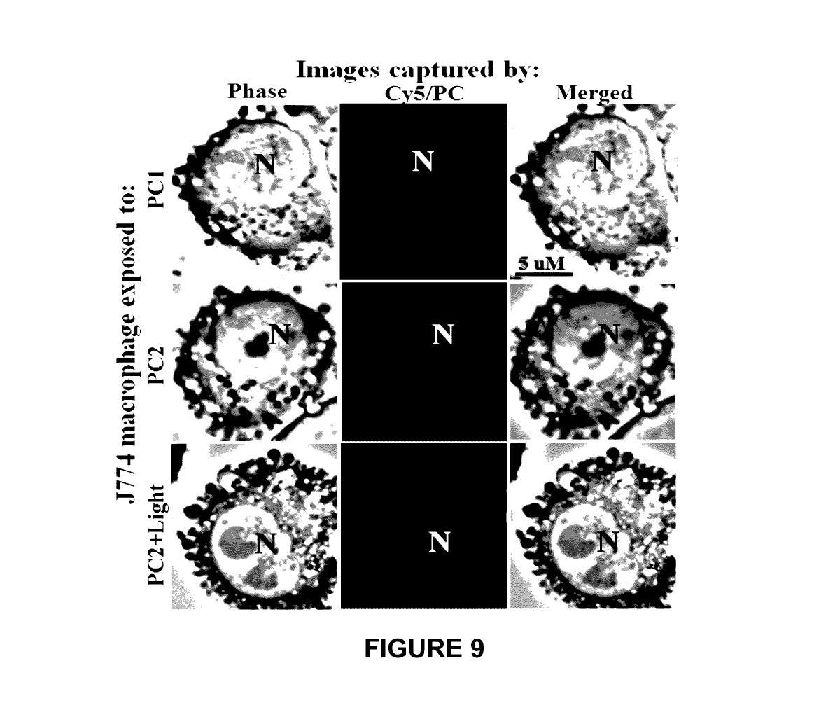

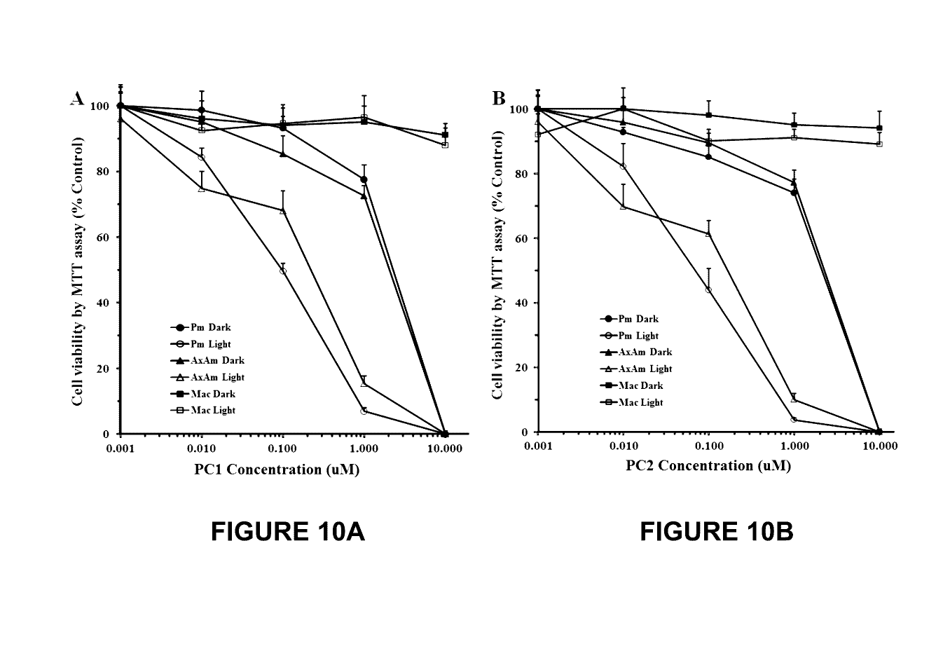

Leishmania are far more sensitive than their host cells or J774 macrophages to PC-mediated photo-inactivation. The J774 cells actively endocytosed PCs 1 and 2 into their endosomes, which accumulated in the peri-nuclear region (FIG. 9). The PC-loaded macrophages remained unchanged morphologically (FIG. 9, 3^{rd} row) and metabolically at all PC concentrations used up to 10 μM, irrespective of exposure to red light (FIGS. 10A and 10B, open and solid squares). The EC₅₀ for these cells thus must be >10 μM for both PCs. Under the same conditions, both Leishmania stages lost their viability to photo-inactivation progressively with increasing concentrations of PCs up to 1 μM (c.f. FIGS. 7A and 7D). Their loss of cell viability was complete at 10 μM with or without red light exposure (FIGS. 10A and 10B, circles and triangles at 10 μM).

The EC₅₀ values of PC1 and PC2 for reducing Leishmania viability were estimated from the graphs presented in FIGS. 10A and 10B. Under the dark conditions, the EC₅₀ values of both PCs appeared to fall somewhere in between 1-10 μM, comparable for both Leishmania stages (FIGS. 10A and 10B, solid triangles and circles). The EC₅₀ values are significantly lowered by red light exposure, varying with PCs and Leishmania stages. The estimated EC₅₀ values of PC1 are ˜0.3 μM and ˜0.1 μM, and those of PC2 are 0.2 μM and 0.07 μM for axenic amastigotes and promastigotes, respectively. Thus, PC2 is 1.5 times more effective than PC1 against both Leishmania stages, while axenic amastigotes are ˜3 times more resistant than promastigotes to both PCs. All EC₅₀ values were estimated from data obtained by the MTT assay—the only method usable to assess the viability of both Leishmania and macrophages. These EC₅₀ values of PCs 1 and 2 for Leishmania are consistent with the data presented in FIGS. 7A and 7D, but appeared higher than those from the fluorescence assays, judging from the data presented in FIGS. 7C-7E.

### DISCUSSION

For photo-inactivation of Leishmania, the amino-PCs used here are 10-40 times more effective, depending on Leishmania stages, than both the endosome-targeting pyridyloxy PCs and mitochondrion-targeting anilinium PSs previously examined, as indicated by comparing the EC50s of PCs 1 and 2 (FIG. 10) versus PC 14 and PC 3.5 (see, e.g., FIG. 2 in Dutta et al., 2011, PLoS One 6:e20786). Even more striking are the host-parasite differential sensitivities to these PCs for photo-inactivation, as reflected in their EC₅₀ values that is in favor of anti-Leishmania PDT greatly by >100 times for PCs 1 and 2 (FIG. 10), but only ˜3 times for PC14 and negative against Leishmania by 10-20 folds for PC3.5. The differences of these PCs in mediating Leishmania photo-inactivation are due neither to the use of different Leishmania spp. (data not shown) nor to their photodynamic properties per se, e.g. quantum yields, but apparently to their differences in the axial substitutions with different ligands. Axial substitution of the Si-PC with 2 symmetrical mono- or di-amino groups (FIG. 1) instead of pyridyloxy groups appears to significantly improve its endocytic bioavailability (cationicity and solubility) to Leishmania cells for photosensitization in aqueous milieu. While the precise mechanisms remain unknown, empirical observations showed that PC1 and PC2 stayed soluble longer in the culture media than the other PCs. Also unknown is the unexpected finding of the significant host-parasite disparity seen in their susceptibility to PC1- and PC2-mediated photo-inactivation under the experimental conditions used.

In summary, the results obtained identify PC1 and PC2 as effective photosensitizers for oxidative photo-inactivation of Leishmania for use as vehicles in drug/vaccine delivery, considering their being up to 40 times more effective than those used previously (see, e.g., Dutta et al, 2011, PLoS One 6:e20786). In addition, the finding that Leishmania are >100 times more sensitive than macrophages to sensitization by PCs 1 and 2 for photo-inactivation suggests their utility for anti-Leishmania PDT.

### Example 5

### Amino-Phthalocyanine (PC1 and PC2)-Mediated Photo-Inactivation of Leishmania for Peptide Delivery for Processing by Antigen-Presenting Cells

J774 Macrophage Processing of Green Fluorescent Protein (GFP) Delivered by Amino-Phthalocyanine (PC)-Photoinactivated Leishmania GFP-Transfectants.

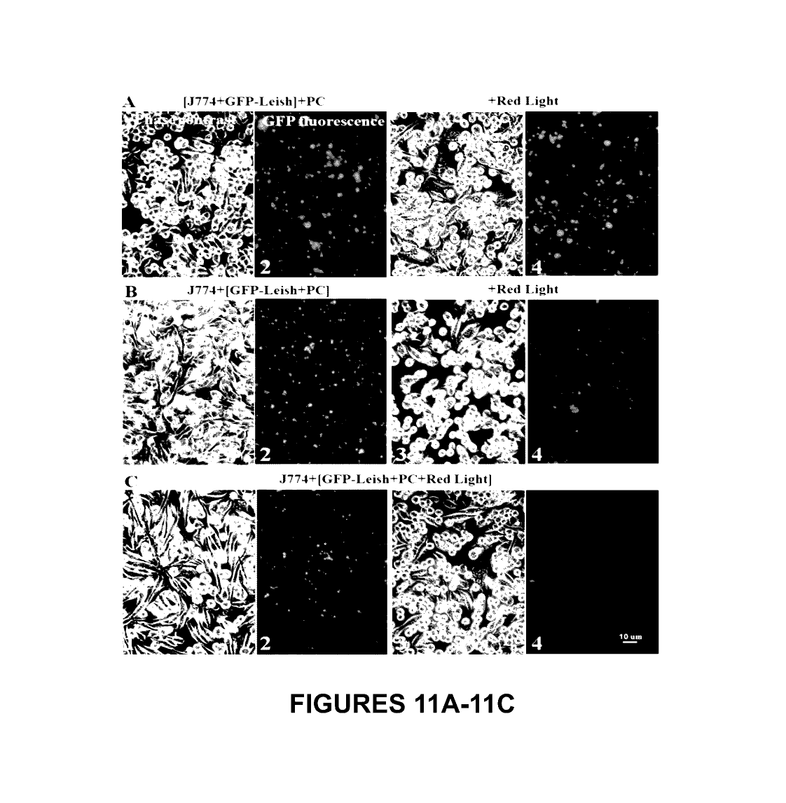

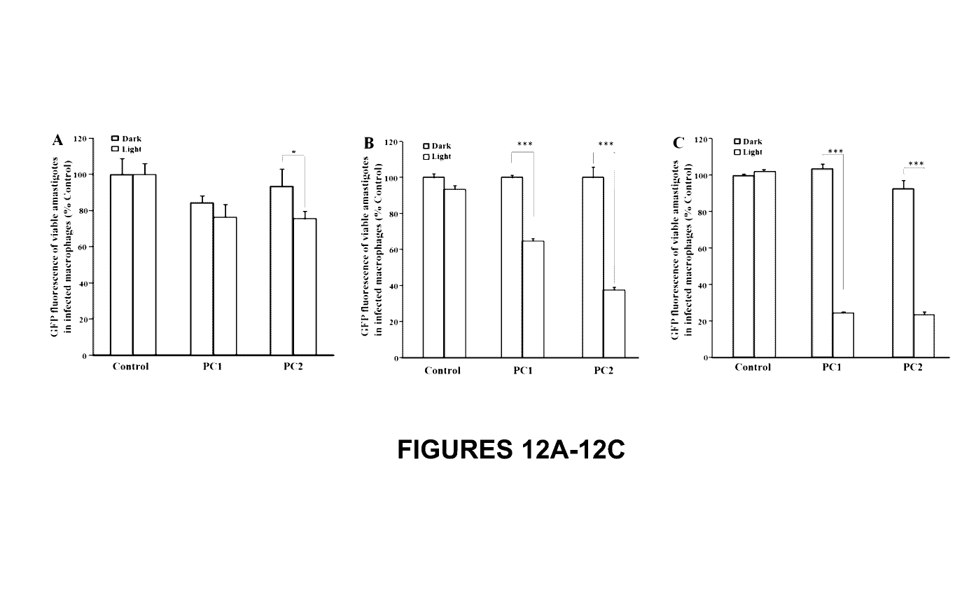

Three different schemes of PC-sensitization and photo-inactivation of Leishmania for peptide vaccine delivery to macrophages (Chang et al., 2016, Parasites & Vectors 9:396) were investigated using GFP as an example. Each scheme began with a mixture of 4×10⁶ J774 cells and 40×10⁶ Leishmania GFP transfectants in 4 ml RPMI1640 with 10% FBS in 25 cm² TC flasks. However, in each scheme, the timing of PC sensitization and red light photo-inactivation was different. In the first scheme, scheme A ([J774+GFP-Leish]+PC+Red Light), GFP-Leish infection was established in J774 cells first (2 days) followed by exposure of the infected cultures to 1 μM PC in the dark overnight and then red light illumination (excitation wavelength=˜600 nm, ˜2 J cm²) (FIG. 11A). In the second scheme, scheme B (results shown in FIG. 11B) (J774+[GFP-Leish+PC]+Red light), the procedure was as in the first scheme, except that GFP-Leish were pre-PC-sensitized before use for infecting J774 cells and illumination was administered after infection. In the third scheme, scheme C (FIG. 11C) (J774+[GFP-Leish+PC+Red light]), J774 cells were loaded with PC-sensitized and photo-inactivated GFP-Leishmania (GFP-Leish). All samples were processed 1 day after illumination (day 4) for microscopy and for GFP fluorescence intensity assays (see FIG. 12). The efficacy of the 3 schemes for GFP delivery was observed to be in the order: A>B>C based on fluorescence intensity associated with the J774 monolayer seen before illumination (see panel 2 of FIGS. 11A-11C). The efficacy of photo-inactivation of GFP-Leish to release GFP for intracellular processing in J774 cells was in the reverse order of: C>B>A (see panel 4 of FIGS. 11A-11C). The integrity of the J774 cells is maintained as monolayers in all cases (see panels 1 and 3 of FIGS. 11A-11C), indicative of selective PC-mediated photo-inactivation of Leishmania to release GFP for processing by J774 cells, as shown by its diminishing fluorescence (panel 2 versus panel 4 of FIGS. 11B-11C).

Quantitation of PC-Mediated Photo-Inactivation of GFP-Leishmania Delivered by Three Different Schemes to J774 Macrophages for Processing.

Samples used were prepared as described for the three different schemes as described above for FIG. 11. Monolayers of J774 cells loaded with PC-sensitized GFP-Leish with and without illumination (FIG. 12, white and black bars) were lysed with 0.5% nonidet-P 40 detergent to release GFP (PC1 and PC2). Controls were simultaneously prepared without PC (FIG. 12, control). Fluorescence intensities of all preparations in triplicate for each were quantitatively determined in a plate reader (FIG. 12A: scheme A; FIG. 12B: scheme B; FIG. 12C: scheme C). The efficacy of GFP-Leish photo-inactivation was in the order of: C>B>A, consistent with the fluorescent microscopy of the same samples shown in FIG. 11. The loss of fluorescence in this assay was indicative of a release of GFP from photo-inactivated GFP-Leish in J774 cells followed by degradative processing of GFP in the phagolysosomes of these antigen-presenting cells. The results also indicate that PC2 is more effective than PC1 for scheme B (FIG. 12B, PC1 versus PC2), and that most effective is pre-PC-sensitized and pre-photoinactivated GFP-Leish, although this is at the expense of GFP delivery by Leishmania (see FIG. 11), as expected. PC1 and PC2 are ˜50 times more effective than PC14-15 used previously for preparing photo-vaccines (Dutta et al, 2011, PLoS One 6:e20786).

Intra-Phagolysosomal Degradation of Oxidatively Photo-Inactivated Leishmania to Release GFP for Processing and Precipitation of PC into Inactive Aggregates.

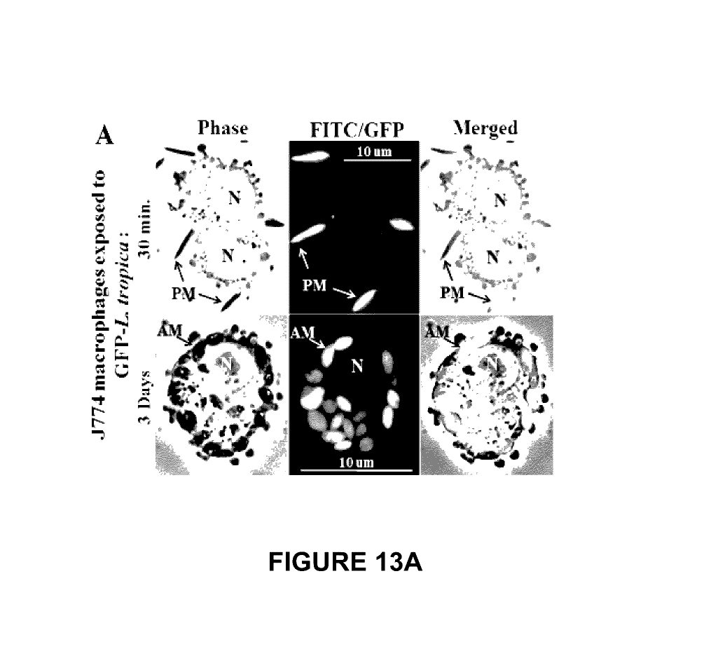

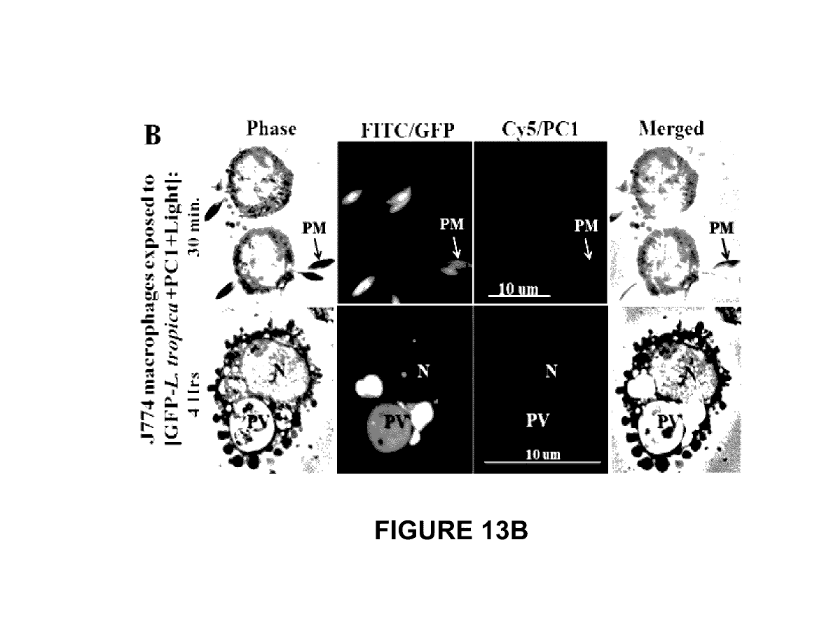

Phase contrast (phase) and fluorescence (FITC/GFP, Cy5, PC1) images are shown in FIG. 13, showing delivery of peptides in the form of GFP to the J774 antigen-presenting cells (APC) by untreated (FIG. 13A) and oxidatively photo-inactivated (FIG. 13B) GFP-transfectants. Schemes A, B, and C, as described above, were used for loading of the J774 cells with GFP transfectants. Scheme A, exposure of J774 cells to untreated GFP-transfectants, resulted in early cell-surface attachment or association, e.g. up to 30 min, and the subsequently intracellular residence of intact Leishmania after further incubation, e.g. 3 days (FIG. 13A). The merged image at day 3 (FIG. 13A) clearly shows delivery of GFP, but not its release by Leishmania, as expected in normal infection. In Scheme B, exposure of J774 cells to GFP-transfectants (FITC/GFP) pre-loaded with PC (Cy5/PC1) followed by red-light illumination produced a different outcome. Observed at the early time point (30 min) were the same images of cell-surface attachment (FIG. 13B). PC-GFP co-localization was observed in J774-attached Leishmania (PM), indicative of PC-loading of their endosomes (30 min, merged image). Upon further incubation for several hours and beyond, oxidatively photo-inactivated Leishmania were seen to lose structural integrity in parasite-containing vacuoles (PV) (Phase), thereby delivering and releasing GFP into the vacuolar space (FITC/GFP) for processing. Aggregates of PC were observed in the same GFP-containing vacuole (4 hrs, FIG. 13B, merged image), suggestive of precipitation of PC to become inactive. APCs are thus not sensitized for photo-inactivation, enabling them to process the released GFP.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

1. A composition for delivering a polypeptide into a mammalian cell, the composition comprising a trypanosomatid that has been treated with a compound of formula (I): 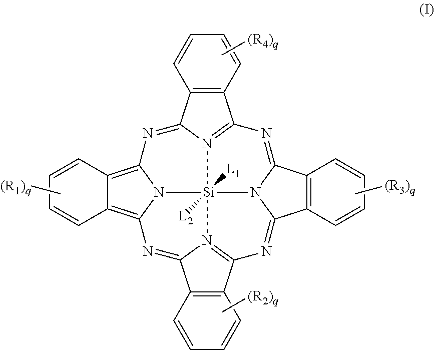
or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
each R is independently —[C₁-C₆ alkylene-O]ₘ—R′ or —[C₁-C₆ alkylene-NR″]ₙ—R′,
each m and n are independently an integer selected from 1 to 20,
each R′ is independently selected from H and C₁-C₆ alkyl,
each R″ is independently selected from H and C₁-C₆ alkyl;
each q is independently an integer selected from 0, 1, and 2; and
R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂;
wherein the trypanosomatid expresses the polypeptide; and
wherein the trypanosomatid is capable of entering the mammalian cell.

2. The composition of claim 1, wherein the trypanosomatid has been transgenically modified to express a cDNA sequence encoding the polypeptide.

3. The composition of claim 1, wherein the polypeptide is a vaccine candidate.

4. The composition of claim 1, wherein the trypanosomatid has been exposed to light to inactivate the trypanosomatid following treatment of the trypanosomatid with the compound of formula (I).

5. The composition of claim 1, wherein the light is white light or red light.

6. The composition of claim 1, wherein the trypanosomatid is a Leishmania sp. trypanosomatid.

7. The composition of claim 1, wherein the mammalian cell is a dendritic cell or a macrophage.

8. The composition of claim 1, wherein each q in formula (I) is an integer selected from 0 and 1.

9. The composition of claim 1, wherein L₁ and L₂ are independently selected from —O(C₁-C₆ alkyl), —O(C₁-C₆ alkenyl), —O(C₁-C₆ alkynyl), and —OR.

10. The composition of claim 1, wherein each R is independently —[C₁-C₆ alkylene-NR″]ₙ—R′, or -[ethylene-NR″]ₙ—R′, or -[propylene-NR″]ₙ—R′; wherein R″ is hydrogen or methyl, or R″ is hydrogen; and wherein R′ is hydrogen or methyl, or R′ is hydrogen, or R′ is methyl.

11. The composition of claim 1, wherein n is an integer selected from 1 to 6, or from 1 to 5, or from 1 to 4, or from 1 to 3, or n is 1, 2, or 3, or n is 1, or n is 2, or n is 3.

12. The composition of claim 1, wherein L₁ and L₂ are independently selected from:

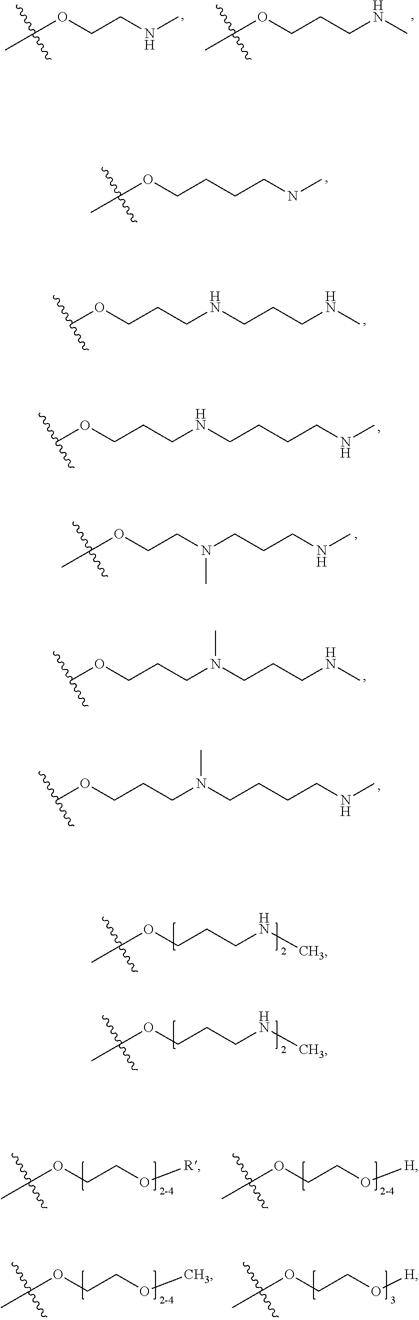

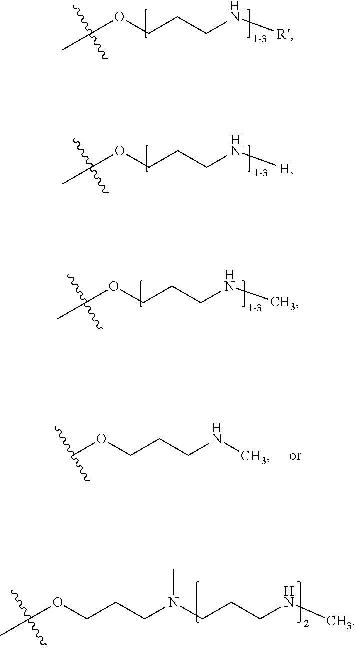

13. The composition of claim 1, wherein L₁ and L₂ are the same.

14. The composition of claim 1, wherein the compound of formula (I) is:

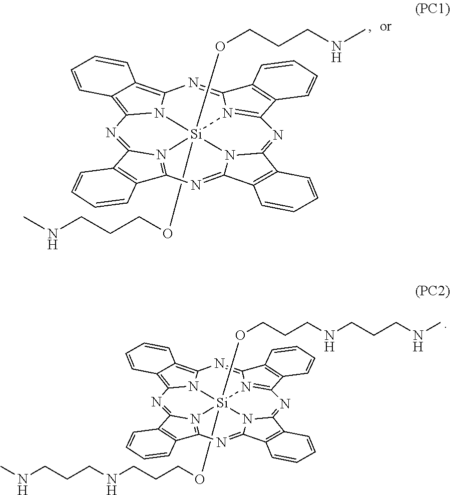

15. A method for delivering a polypeptide to a cell of a mammalian subject, the method comprising:
(a) providing a trypanosomatid that expresses the polypeptide;
(b) treating the trypanosomatid with a photosensitizer to produce a carrier, wherein the photosensitizer is a compound of formula (I): 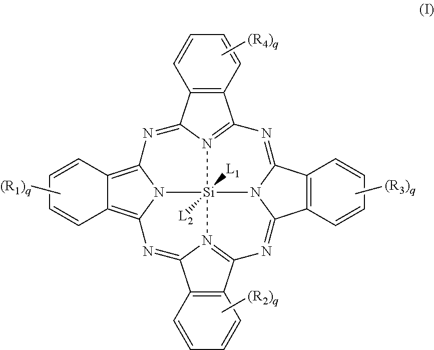
or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
each R is independently —[C₁-C₆ alkylene-O]ₘ—R′ or —[C₁-C₆ alkylene-NR″]ₙ—R′,
each m and n are independently an integer selected from 1 to 20,
each R′ is independently selected from H and C₁-C₆ alkyl,
each R″ is independently selected from H and C₁-C₆ alkyl;
each q is independently an integer selected from 0, 1, and 2; and
R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂;
(c) contacting the cell with the carrier by administering the carrier to the mammalian subject; and
(d) exposing the subject to light to inactivate the carrier.

16. A method for delivering a polypeptide to a cell of a mammalian subject, the method comprising:
(a) providing a trypanosomatid that expresses the polypeptide;
(b) treating the trypanosomatid with a photosensitizer to produce a carrier, wherein the photosensitizer is a compound of formula (I): 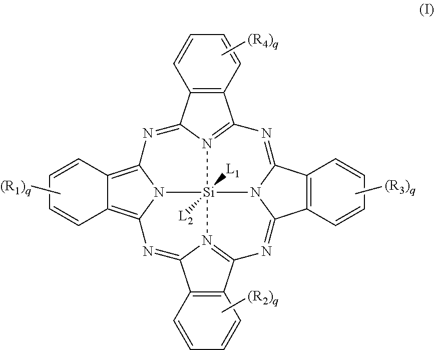
or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
each R is independently —[C₁-C₆ alkylene-O]ₘ—R′ or —[C₁-C₆ alkylene-NR″]ₙ—R′,
each m and n are independently an integer selected from 1 to 20,
each R′ is independently selected from H and C₁-C₆ alkyl,
each R″ is independently selected from H and C₁-C₆ alkyl;
each q is independently an integer selected from 0, 1, and 2; and
R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂;
(c) exposing the carrier to light to inactivate the carrier; and
(d) contacting the cell with the inactivated carrier by administering the carrier to the mammalian subject.

17. The method of claim 16, wherein the trypanosomatid has been transgenically modified to express a cDNA sequence encoding the polypeptide.

18. The method of claim 16, wherein the polypeptide is a vaccine candidate.

19. The method of claim 16, wherein the light is white light or red light.

20. The method of claim 16, wherein the trypanosomatid is a Leishmania sp. trypanosomatid.

21. The method of claim 16, wherein the cell is a dendritic cell or a macrophage.

22. A method of deactivating a trypanosomatid, the method comprising
(a) treating a live trypanosomatid with a compound of formula (I): 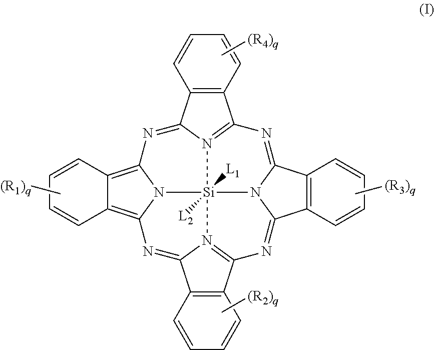
or an acceptable salt thereof,
wherein L₁ and L₂ are independently selected from —O(C₁-C₆ alkyl), —O(C₁-C₆ alkenyl), —O(C₁-C₆ alkynyl), —NH(C₁-C₆ alkyl), —N(C₁-C₆ alkyl)₂, and —OR,
wherein
each R is independently —[C₁-C₆ alkylene-O]ₘ—R′ or —[C₁-C₆ alkylene-NR″]ₙ—R′,
each m and n are independently an integer selected from 1 to 20,
each R′ is independently selected from H and C₁-C₆ alkyl,
each R″ is independently selected from H and C₁-C₆ alkyl;
each q is independently an integer selected from 0, 1, and 2; and
R₁, R₂, R₃, and R₄ are independently selected from halogen, C₁-C₆ alkyl, C₁-C₆ alkenyl, C₁-C₆ alkynyl, —NH(C₁-C₆ alkyl), —N(C₁-C₆ alkyl)₂, C₁-C₆ alkoxy, C₁-C₆ aryloxy, C₁-C₆ heteroaryloxy, and polyalkylene oxide, each optionally substituted with halogen, C₁-C₆ alkyl, —OH, C₁-C₆ alkoxy, —NH₂, —NH(C₁-C₆ alkyl), —N(C₁-C₆ alkyl)₂; and
(b) exposing the treated trypanosomatid to light in the presence of oxygen to deactivate the trypanosomatid.

23. The method of claim 22, wherein the light is white light or red light.

24. The method of claim 22, wherein the trypanosomatid is Leishmania.

25. The method of claim 16, wherein each q in formula (I) is an integer selected from 0 and 1.

26. The method of claim 16, wherein L₁ and L₂ are independently selected from —O(C₁-C₆ alkyl), —O(C₁-C₆ alkenyl), —O(C₁-C₆ alkynyl), and —OR.

27. The method of claim 16, wherein each R is independently —[C₁-C₆ alkylene-NR″]ₙ—R′, or -[ethylene-NR″]ₙ—R′, or -[propylene-NR″]ₙ—R′; wherein R″ is hydrogen or methyl, or R″ is hydrogen; and wherein R′ is hydrogen or methyl, or R′ is hydrogen, or R′ is methyl.

28. The method of claim 16, wherein n is an integer selected from 1 to 6, or from 1 to 5, or from 1 to 4, or from 1 to 3, or n is 1, 2, or 3, or n is 1, or n is 2, or n is 3.

29. The method of claim 16, wherein L₁ and L₂ are independently selected from:

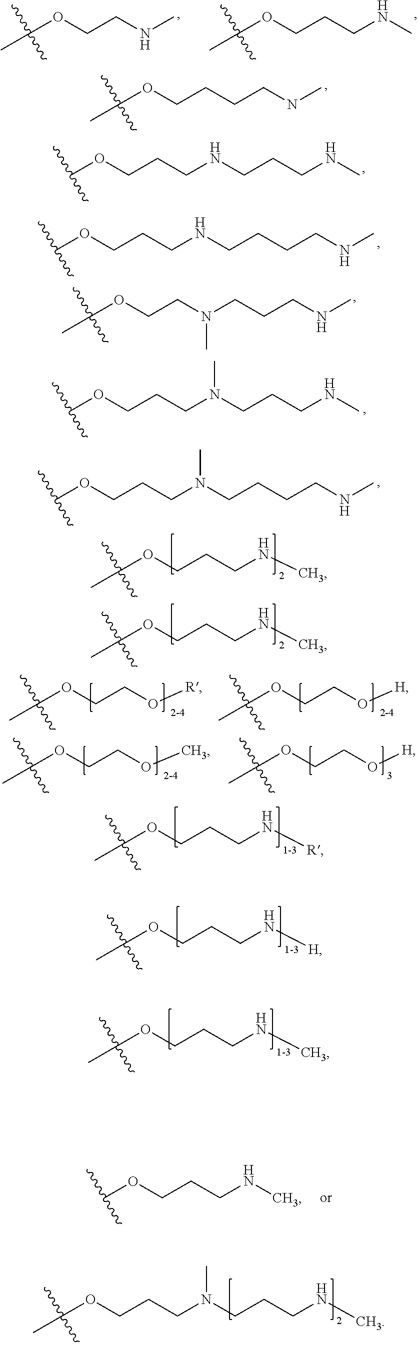

30. The method of claim 16, wherein L₁ and L₂ are the same.

31. The method of claim 16, wherein the compound of formula (I) is:

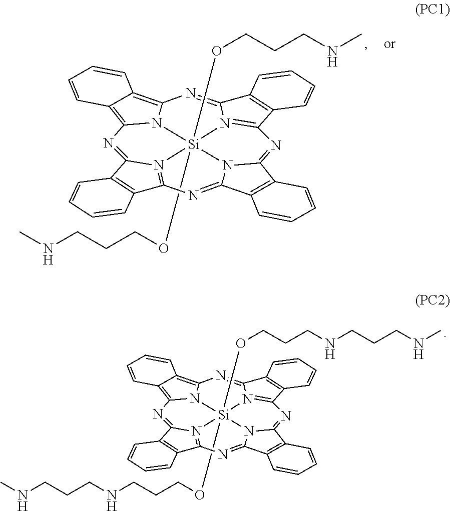

32. A vaccine composition comprising the deactivated trypanosomatid prepared by the method of claim 22 and a suitable pharmaceutical carrier.

33. A method for inducing an immune response against a trypanosomatid in a mammalian subject comprising administering to the subject the vaccine composition of claim 32.

34. A method for inducing an immune response against Leishmania in a subject comprising administering to the subject the vaccine according to claim 32, wherein the trypanosomatid is Leishmania.
